(19) United States
(12) Patent Application Publication
(10) Pub. No.: US 2011/0009608 A1
(43) Pub. Date: Jan. 13, 2011

(54) AUTOMATIC REFINING APPARATUS, MULTI-WELL PLATE KIT AND METHOD FOR EXTRACTING HEXANE FROM BIOLOGICAL SAMPLES

(75) Inventors: Jong-Hoon Kim, Daejeon (KR); Jong Kab Kim, Daejeon (KR); Yang Won Lee, Daejeon (KR); Han Oh Park, Daejeon (KR)

Correspondence Address: CANTOR COLBURN LLP, 20 Church Street, 22nd Floor, Hartford, CT, 06103, US

(73) Assignee: BIONEER CORPORATION, Daejeon (KR)

(21) Appl. No.: 12/920,475

(22) PCT Filed: Apr. 8, 2009
(86) PCT No.: PCT/KR09/01804

§ 371 Date: Sep. 1, 2010

(30) Foreign Application Priority Data
Apr. 9, 2008 (KR) 10-2008-0032904

(51) Int. Cl. C07H 21/00; B03C 1/02; B03C 1/30

(52) U.S. Cl. 536/25.41; 210/222; 210/175; 210/209

(57) ABSTRACT

The present invention relates to an automatic refining apparatus for separating target materials from a plurality of biological sample solutions by using magnetic particles to which the magnetic particles are to be reversibly coupled, and to a multi-well plate kit for use in the automatic refining apparatus. Further, the present invention relates to a method for extracting nucleic acids from biological samples by using the above-described automatic refining apparatus. The present invention can be used in the automatic separation of nucleic acid, protein, and the like from biological samples.

### TECHNICAL FIELD

The present invention relates to an automatic refining apparatus for separating target materials from a plurality of biological samples by using magnetic particles to which the magnetic particles are to be reversibly coupled, and to a multi-well plate kit for use in the automatic refining apparatus.

Further, the present invention relates to a method for extracting nucleic acids from biological samples by using the above-described automatic refining apparatus.

### BACKGROUND ART

A variety of techniques have been developed to separate nucleic acids, protein, or the like from biological samples. Traditionally, precipitation, liquid-phase extraction, electrophoresis, chromatography, and so forth have been used frequently. Recently, solid-phase extraction was developed for a simpler manipulation. In the solid-phase extraction technique, highly selective solid materials or solid particles with highly selective ligands attached thereto are used. According to this method, a biological sample is dissolved in a solution allowing selective adhesion of target materials. After the target materials are attached on the solid materials, the solid materials are separated from the solution and then the liquid adhering to the solid is washed off to remove other impurities, so that the desired target materials are separated from the solution. In the solid-phase extraction method, a column packed with fine solid particles or a filter membrane is used. Fine particles with large surface area are used to increase adhesion volume, and the filter membrane is used when the volume of the sample is small. However, the use of the fine particles or filter membrane is problematic in that the solution flows very slowly through small pores. Thus, in order to increase the solution flow rate, a centrifuge is used to increase centrifugal force or a pressure difference is made by applying or reducing pressure. However, the method based on centrifugation is not well suited to automation. Although the method based on pressure difference can be relatively simply automated, the solution flow rate may be different between samples when a plurality of samples are handled.

This problem can be solved by using fine magnetic particles with large surface area. By quickly adhering biochemical substances on suspended solids, making the magnetic particles with the target materials bound aggregate by applying a magnetic field and then removing the solution, the target materials can be separated conveniently. This method has been developed since 1970s (U.S. Pat. No. 3,970,518; U.S. Pat. No. 3,985,649). Because this method can be easily automated, various apparatuses for separating target material using magnetic particles have been developed.

The method of separating target materials from a biochemical solution using magnetic particles consists of the following 3 stages: adhesion of the target materials; removal of the solution and washing; and detachment of the target materials. This procedure is necessary for automation. Although seemingly complicated, the procedure may be classified into two operations depending on the state of the magnetic particles. One is to suspend the magnetic particles uniformly in the solution, and the other is to aggregate the magnetic particles suspended in the solution.

In order to uniformly suspend the magnetic particles in the solution, the container holding the solution may be shaken strongly to form a vortex. Optionally, the solution may be stirred using rods to form the vortex. In addition, the solution may be repeatedly sucked in and out to form the vortex.

In order to aggregate the magnetic particles suspended in the solution, a magnetic field is applied. The magnetic field may be either from a permanent magnet or from an electromagnet. In general, the permanent magnet has the advantage that, unlike the electromagnet, a strong magnetic field may be produced without heat generation. However, since the permanent magnet is incapable of switching on/off of magnetic flux as in the electromagnet, a physical switching between the solution of the magnetic particles and the magnet is necessary. This makes automation difficult.

The location where the magnetic particles are aggregated changes depending on the location where the magnetic field is applied. Since the location where the magnetic particles are aggregated is important in effective removal of the solution, techniques have been developed thereabout. Separation apparatuses using magnetic particles have been developed mainly for diagnostic devices based on antigen-antibody reactions and nucleic acid extraction devices. A method of attaching magnetic particles on the bottom of a 96-well plate and then suspending them again was developed by Pasteur Sanofi Diagnostic (U.S. Pat. No. 5,558,839). This method is associated with the problem that the magnetic particles at the bottom may be lost while the solution is removed. To solve this problem, a method of providing a magnet beside a container, rotating the magnet or the container to induce suspension and then stopping the rotation so that the magnetic particles are aggregated on the wall of the container was developed (WO 96/26011). Hitachi developed a system whereby aggregation and suspension are possible using a constant magnetic field and an alternating magnetic field (U.S. Pat. No. 5,770,461). According to this method, magnetic particles are attached on the wall of a tube using a constant magnetic field and, after washing, are suspended using an alternating magnetic field. Amersham International plc developed a system whereby magnetic particles are attached on the inner wall of a tube in circular shape by moving a doughnut-shaped magnet vertically relative to a container to switch a magnetic field (U.S. Pat. No. 5,897,783). According to the aforesaid methods, magnetic particles are aggregated in a reaction container and, after removing the solution and then adding a fresh solution, the magnetic particles are suspended again.

In contrast to these methods, Labsystems developed a method of moving magnetic particles between reaction containers holding solutions to suspend them. The system is equipped with a rod capable of moving upward and downward like a fishing rod and a rod pocket. A permanent magnet is provided at the lower end of the rod, and the rod pocket is a plastic rod allowing penetration of the magnetic field and prevents the rod from contacting the solution (U.S. Pat. No. 6,040,192). Operation is as follows. In the state where the magnet rod is outside the rod pocket, the rod pocket is put in a reaction solution containing the magnetic particles and is moved up and down so that reaction with the magnetic particles may occur. Then, the magnet rod is inserted in the rod pocket and the magnetic particles are made to attach on the surface of the magnet rod pocket by the magnetic field from the magnet rod. Then, the magnetic particles with the wanted target material attached are moved to the next solution along with the magnet rod and the rod pocket. Thereafter, the magnet rod is pulled from the rod pocket to remove the magnetic field, and the rod pocket is moved up and down so that the magnetic particles are suspended in the new solution. An automatic nucleic acid extractor operating by the same principle was developed by Bionex (KR 10-0483684). Like the '192 patent, it attaches magnetic particles to a rod pocket accommodating various magnet rods and extracts nucleic acids by moving it to another solution and suspending the magnetic particles. Both techniques by Labsystems and Bionex are restricted in treating a plurality of samples because they are designed to treat samples in a row. Thus, a technique of using rods and rod pockets arranged in a 2-dimensional array for automatic extraction of a plurality of samples, e.g. in a 96-wellplate, was developedbyCoreBio System (KR 10-0720044). According to the three techniques, respective solutions are located at specific positions and, after selective attachment and washing, the target materials are separated from the magnetic particles in the last solution to separate the wanted substance. Accordingly, the cumbersome procedure of transferring the sample from the last solution to a container for storage is necessary. Further, since the target materials are transferred being attached on the rod pocket, care is needed during initial setting to avoid surface contamination. Also, it is tiresome to set up the individual rod pockets and solution cartridges.

There is a flexible method allowing the transfer of both the magnetic particles and the solution as desired. Labsystems' U.S. Pat. No. 5,647,994 (priority date: Jun. 21, 1993) describes several methods of separating magnetic particles using disposable pipettes. It is a prior art preceding U.S. Pat. No. 5,702,950 or U.S. Pat. No. 6,187,270 with respect to attachment of magnetic particles to the pipette. A magnetic field is applied to the pipette by disposing a doughnut-shaped magnet around the pipette so that the pipette penetrates the magnet. Then, the magnetic field is switched by moving the magnet up and down. Alternatively, the magnetic field may be switched by moving a magnetic field shielding metal between the doughnut-shaped magnet and the pipette which are fixed. The '994 patent also suggests suspending and collecting the magnetic particles by moving a magnet rod, which is protected by a rod pocket from contact with the solution at the center, up and down for the first time. This precedes U.S. Pat. No. 6,040,192 owned by the same company. In claims 1 and 2, the '994 patent discloses a method of separating magnetic particles from a first solution containing the magnetic particles and of transferring the magnetic particles to a second solution and a separating means therefor, respectively. Claim 1 claims a method comprising: providing a tubular member defining a separation chamber serially connected to a jet channel, wherein the jet channel defines a flow port at an end of the tubular member and the jet channel has a diameter smaller than that of the separation chamber; providing a magnet element for generating a magnetic field; drawing a first solution through the jet channel via the flow port into the separation chamber; disposing the magnet element at one of a first location adjacent to an outer side of the separation chamber and a second location within the separation chamber; activating the magnet element such that magnetic particles under the influence of the magnetic field of the magnet element will collect on the side of the first solution onto one of the inner side of the separation chamber when disposed in the first location and a collection surface of the magnet element when disposed in the second location; removing the first solution through the jet channel via the flow port after activating the magnet element; drawing the second solution into a container through the jet channel via the flow after removing the first solution; and deactivating the magnet element such that the magnetic field of the magnet element no longer keeps the magnetic particles on one of the inner surface of the separation chamber when disposed in the first location and on the collection surface when disposed in the second location after drawing the second solution. Claim 2 claims a separating means comprising: a tubular member having a first portion defining a separation chamber serially connected to a jet channel, wherein the jet channel defines a flow port at an end of the tubular member and the jet channel has a diameter smaller than that of the separation chamber; a magnet element disposed at one of a first location adjacent to an outer side of the separation chamber and a second location within the separation chamber, wherein the magnet element is adapted to be brought into such a state that magnetic particles under the a magnetic field will keep the magnetic particles when disposed in the first location, or into such a state that the magnetic field no longer keeps the magnetic particles when disposed in the second location; wherein the tubular member has a second portion defining a cylindrical channel serially connected to the separation chamber remote from the jet channel, the cylindrical channel receiving a movable piston for drawing liquid into the separation chamber and for removing the liquid from the separation chamber. Precision System Science's U.S. Pat. No. 5,702,950 (priority date: Jun. 14, 1994) discloses a method for attracting and releasing a magnetic material using magnetic particles for use in an immunochemical analyzer. An analyzer using the technique was filed as a divisional application (U.S. Pat. No. 6,231,814). Basically, the technique is on the same principle as U.S. Pat. No. 5,647,994 whereby the magnetic particles are attached to the pipette. The difference is that a magnetic field is controlled by moving a magnet close to or away from the pipette at one side. The method for attracting and releasing a magnetic material comprises: providing a pipette device having a liquid suction line including a liquid inlet for sucking a liquid containing magnetic particles from a container and discharging the liquid, and a magnet body or magnet bodies being detachably fitted to an external surface of the liquid suction line of the pipette device; the pipette device providing attracting/releasing control by absorbing and maintaining the magnetic material contained in the liquid and attracted to the liquid suction line due to a magnetic field generated by the magnet body or bodies on an internal surface of the liquid suction line and by releasing the magnetic material from the liquid suction line by means of interrupting effect by the magnetic field so that the magnetic material is discharged together with the liquid outside of the liquid suction line through the liquid inlet.

Roche Diagnostics proposed a device for separating magnetic particles from a liquid whereby a permanent magnet is approached near a disposable tip so as to attach the magnetic particles (U.S. Pat. No. 6,187,270). The device comprises a pipette connected to a pump, a magnet, and a moving means for causing relative movement of the pipette toward or away from the magnet. Claim 1 of the '270 patent claims a device for separating magnetic microparticles from a liquid, comprising: a pipette having an inner wall, the pipette containing a liquid containing magnetic microparticles therein, wherein the pipette is rotatable along a longitudinal direction; a pump connected to the pipette; a magnet exterior of the pipette and locatable to apply a magnetic field to attach the magnetic microparticles on the inner wall of the pipette; and a moving means for causing relative movement of the pipette and the magnet to move at least one of them toward each other. Claim 2 claims a device for separating magnetic microparticles from a liquid, comprising: a pipette having an inner wall, the pipette containing a liquid containing magnetic microparticles therein; a pump connected to the pipette; a magnet exterior of the pipette and locatable to apply a magnetic field to attach the magnetic microparticles on the inner wall of the pipette; and a moving means for causing relative movement of the pipette and the magnet to move at least one of them toward each other, wherein the magnet is movable along a longitudinal direction of the pipette. Another independent claim also relates to collect and release magnetic microparticles through relative movement of the pipette and the magnet.

As described, the above-described methods separate magnetic microparticles from a solution by attaching them to a tip of a disposable pipette and then suspending them in another solution. However, they are limited in handling a plurality of samples conveniently and quickly.

### DETAILED DESCRIPTION OF THE INVENTION

### Technical Problem

The present invention relates to an automatic refining apparatus for separating wanted materials from a plurality of biological samples conveniently and quickly. Although a lot of apparatuses have been developed whereby biological samples are treated using magnetic particles, most of them have a large size because of complicated structure. In addition, they are expensive and difficult to use. In particular, since one solution block and one pipette are used for one sample in an automatic refining apparatus using magnetic nanoparticles, there is a problem in cost and setting time when a plurality of samples are handled. Further, since the purified nucleic acid has to be transferred from the purification block to a storage container after the refining is completed, there is little advantage over manual purification of nucleic acids. Thus, those apparatuses are not used widely and, in most laboratories, nucleic acids are purified manually, for example, by using centrifuges. As a result, the reproducibility of nucleic acid purification is low and a lot of high-quality human resources are wasted in purification of nucleic acids. To solve this problem, the present invention is directed to providing a multi-well plate kit and an automatic refining apparatus allowing fast, convenient and economical purification of nucleic acids, wherein the kit is configured with multi-well blocks containing different solutions including magnetic particles so that reagents can be loaded quickly and conveniently, a plurality of pipettes are provided in two rows to allow treatment of a plurality of samples while reducing the apparatus size, a uniform magnetic field is applied to the pipettes in each row during the same time interval, and the whole process from the insertion of the pipettes to the transfer of the final product to the sample storage container is automated.

The present invention is also directed to providing a method for extracting nucleic acids, capable of preventing decrease in activity or sensitivity of enzymes used in polymerase chain reaction, real-time polymerase chain reaction, sequencing, or the like, which may be caused by direct or indirect reactions with alcohols eluted along with the nucleic acids.

### Technical Solution

The present invention provides an automatic refining apparatus for separating target materials from a plurality of biological samples by using magnetic particles to which the magnetic particles are to be reversibly coupled, comprising: a pipette block having a plurality of pipettes mounted in at least two rows for sucking and discharging biological samples including target materials into and out of the plurality of pipettes; a fixing body supporting the pipette block; a magnetic field application unit for applying and releasing a magnetic field to the pipettes of each row mounted on the pipette block; a pipette block upward/downward moving means moving the pipette block upward and downward; and a pipette block forward/backward moving means moving the pipette block forward and backward.

The pipette block may comprise: a piston fixing plate wherein a plurality of pistons are provided in two rows; a piston moving means moving the piston fixing plate upward and downward; a piston guiding unit having piston guide holes guiding the upward and downward movement of the a plurality of pistons; and pipette mounting units extending below the piston guiding unit in two rows so as to be engaged with the inner periphery of the plurality of pipettes arranged in two rows and having a plurality of connecting holes respectively communicating with the piston guide holes. Engagement rings may be provided at the outer periphery of the pipette mounting units so that the pipette mounting units may be engaged with the inner periphery of the pipettes.

The pipette block may comprise: a piston guiding unit supporting plate supporting the piston guiding unit; a guide rod protruding from an upper surface of the piston guiding unit supporting plate and guiding the upward and downward movement of the piston fixing plate; and a pipette separating unit contacting a lower surface of the piston fixing plate and separating the plurality of pipettes mounted on the pipette mounting units. The pipette separating unit may comprise: a detachable upper plate provided above the piston guiding unit through which the plurality of pistons penetrate; a detachable lower plate provided below the piston guiding unit supporting plate and separating the plurality of pipettes mounted on the pipette mounting units by pressing the upper end thereof downward; an up/down connecting rod connecting the detachable upper plate and the detachable lower plate with a gap; a protruding rod provided on an upper surface of the detachable lower plate and protruding above the piston guiding unit supporting plate via through-holes formed on the piston guiding unit supporting plate; and a spring the lower end of which being supported by the upper surface of the piston guiding unit supporting plate and the upper end of which being supported by an upper end of the protruding rod so as to exert an elastic force so that the detachable lower plate is fastened to the piston guiding unit supporting plate.

The piston moving means may comprise: a piston driving motor supporting plate supported by the guide rod and having a piston driving motor mounted thereon; and a piston control screw moving upward and downward by the piston driving motor and the lower end of which being connected to the piston fixing plate. The magnetic field application unit may comprise: a first row magnet mounting unit having a magnet for applying a magnetic field to the pipettes mounted on the first row of the pipette block; a second row magnet mounting unit having a magnet for applying a magnetic field to the pipettes mounted on the second row of the pipette block; a first row magnet mounting unit moving means for controlling a distance between the magnet of the first row magnet mounting unit and the pipettes mounted on the first row of the pipette block; and a second row magnet mounting unit moving means for controlling a distance between the magnet of the second row magnet mounting unit and the pipettes mounted on the second row of the pipette block, wherein the strength and duration of the magnetic field applied to the pipettes of the first row by the first row magnet mounting unit and the first row magnet mounting unit moving means are the same as the strength and duration of the magnetic field applied to the pipettes of the second row by the second row magnet mounting unit and the second row magnet mounting unit moving means.

The first row magnet mounting unit comprises a first row middle plate located by the first row magnet mounting unit moving means between neighboring pipettes among the pipettes of the first row and having a magnet mounted thereon and a first row end plate located by the first row magnet mounting unit moving means outside of a pipette located at the side end among the pipettes of the first row and having a magnet mounted thereon, and the second row magnet mounting unit comprises a second row middle plate located by the second row magnet mounting unit moving means between neighboring pipettes among the pipettes of the second row and having a magnet mounted thereon and a second row end plate located by the second row magnet mounting unit moving means outside of a pipette located at the side end among the pipettes of the second row and having a magnet mounted thereon. The first row middle plate and the first row end plate may have through-holes provided in a direction parallel to the row direction of the pipettes of the first row to allow the mounting of the magnets, and the second row middle plate and the second row end plate may have through-holes provided in a direction parallel to the row direction of the pipettes of the second row to allow the mounting of the magnets.

The first row magnet mounting unit moving means may comprise a first row gear connected to the pipette block and rotated by a magnet mounting unit motor and a first row rotation shaft rotated by the rotation of the first row gear. The second row magnet mounting unit moving means may comprise a second row gear connected to the pipette block and rotated in the opposite direction of the first row gear as being engaged with the first row gear and a second row rotation shaft rotated by the rotation of the second row gear. The first row magnet mounting unit may be radially connected to the first row rotation shaft so as to rotate and the second row magnet mounting unit may be radially connected to the second row rotation shaft so as to rotate.

The pipette block may be mounted on the fixing body so as to be movable upward and downward. The pipette block upward/downward moving means may comprise an up/down movement motor provided at the fixing body and an up/down movement screw rotated by the up/down movement motor so as to move a fixing nut fixed to the pipette block upward and downward, and the pipette block forward/backward moving means may comprise a forward/backward movement supporting rod supporting the fixing body so as to be movable forward and backward and a forward/backward moving belt attached to the fixing body so as to move the fixing body forward and backward. The automatic refining apparatus may comprise a base plate below the fixing body, the base plate having a multi-well plate kit, a pipette rack insertably holding the plurality of pipettes mounted on the pipette block in two rows, a sample storage tube rack insertably holding a plurality of sample storage tubes for storing the purified sample in two rows, and a waste bottle for holding waste solution discarded from the plurality of pipettes mounted on the pipette block mounted thereon. The base plate may have a high-temperature reaction block for heating a plurality of high-temperature reaction tubes insertably held in two rows mounted thereon. The automatic refining apparatus may comprise a casing accommodating the pipette block, the fixing body, the pipette block upward/downward moving means, the pipette block forward/backward moving means and the base plate, wherein a UV lamp or an ozone generator for sterilization is provided in the casing.

The present invention also provides a multi-well plate kit used in the automatic refining apparatus, comprising a plurality of unit wells arranged in two neighboring rows and a film sealing an upper end of the plurality of unit wells, wherein solutions for extraction of target materials are contained in the unit wells excluding at least one unit well (s) such that the same solution is contained in the same unit well. The solution contained in one of the sealed unit wells may be an aqueous suspension wherein magnetic particles are suspended and the magnetic particles suspended in the aqueous suspension may be spherical magnetic particles coated with silica.

The present invention further provides a method for extracting nucleic acids from biological samples using the automatic refining apparatus, comprising: mixing a biological sample with a cell lysis solution contained in a well of the multi-well plate kit using the pipette; mixing the sample mixed with the cell lysis solution with a coupling solution contained in a well of the multi-well plate kit using the pipette; mixing the mixture with the coupling solution with an aqueous suspension of magnetic particles contained in a well of the multi-well plate kit using the pipette; in the state where the mixture with the coupling solution is held in the pipette, applying a discharge pressure to the pipette such that the mixture is discharged from the pipette while applying a magnetic field to the pipette at the same time such that the magnetic particles and materials attached to the magnetic particles are not discharged by the discharge pressure but remain in the pipette; releasing the magnetic field and mixing the magnetic particles and the materials attached to the magnetic particles with a washing solution containing alcohol contained in a well of the multi-well plate kit so as to remove impurities other than nucleic acids from the magnetic particles; in the state where the mixture with the washing solution is held in the pipette, applying a discharge pressure to the pipette such that the mixture is discharged from the pipette while applying a magnetic field to the pipette at the same time such that the magnetic particles with nucleic acids attached thereto are not discharged by the discharge pressure but remain in the pipette; releasing the magnetic field and injecting the magnetic particles with the nucleic acids attached thereto into a high-temperature reaction tube on a high-temperature reaction block so as to remove the alcohol from the washing solution remaining on the magnetic particles; mixing a nucleic acid eluent contained in a well of the multi-well plate kit with the magnetic particles held in the high-temperature reaction tube using the pipette so as to elute the nucleic acids; and, in the state where the nucleic acid eluent including the nucleic acids eluted from the magnetic particles and the magnetic particles are held in the pipette, applying a discharge pressure to the pipette such that the eluent including the nucleic acids is discharged from the pipette while applying a magnetic field to the pipette at the same time such that the magnetic particles are not discharged by the discharge pressure but remain in the pipette.

The present invention further provides a method for extracting nucleic acids from biological samples using the automatic refining apparatus, comprising: mixing a biological sample contained in a well of the multi-well plate kit with a cell lysis solution contained in a well of the multi-well plate kit using the pipette; mixing the cell lysis solution and the biological sample with lysed cells with a coupling solution contained in a well of the multi-well plate kit using the pipette; mixing the mixture with the coupling solution with an aqueous suspension of magnetic particles contained in a well of the multi-well plate kit using the pipette; in the state where the mixture with the coupling solution is held in the pipette and located above the waste bottle, applying a discharge pressure to the pipette by a downward movement of a piston such that the mixture with the coupling solution is discharged from the pipette while applying a magnetic field to the pipette at the same time using a magnet mounting unit such that the magnetic particles and materials attached to the magnetic particles are not discharged by the discharge pressure but remain in the pipette; releasing the magnetic field and mixing the magnetic particles and the materials attached to the magnetic particles with a washing solution containing alcohol contained in a well of the multi-well plate kit so as to remove impurities other than nucleic acids from the magnetic particles; in the state where the mixture with the washing solution is held in the pipette and located above the waste bottle, applying a discharge pressure to the pipette by a downward movement of the piston such that the mixture with the washing solution is discharged from the pipette while applying a magnetic field to the pipette at the same time using the magnet mounting unit such that the magnetic particles with nucleic acids attached thereto are not discharged by the discharge pressure but remain in the pipette; releasing the magnetic field and injecting the magnetic particles with the nucleic acids attached thereto into a high-temperature reaction tube so as to remove the alcohol from the washing solution remaining on the magnetic particles; mixing a nucleic acid eluent contained in a well of the multi-well plate kit with the magnetic particles held in the high-temperature reaction tube using the pipette so as to separate the nucleic acids; and in the state where the nucleic acid eluent including the nucleic acids separated from the magnetic particles and the magnetic particles are held in the pipette and located above the sample storage tube, applying a discharge pressure to the pipette by a downward movement of the piston such that the nucleic acid eluent including the nucleic acids is discharged from the pipette while applying a magnetic field to the pipette at the same time using the magnet mounting unit such that the magnetic particles are not discharged by the discharge pressure but remain in the pipette.

The removal of the alcohol from the washing solution remaining on the magnetic particles may comprise: in the where the magnetic particles are held in the pipette, injecting alcohol contained in a well of the multi-well plate kit to the pipette by an upward movement of the piston so as to allow easy injection of the magnetic particles into the high-temperature reaction tube; and injecting the alcohol injected from the well of the multi-well plate kit to the pipette to the high-temperature reaction tube along with the magnetic particles with the nucleic acids thereto. The removal of the alcohol from the washing solution remaining on the magnetic particles may comprise, in the where the magnetic particles with the nucleic acids thereto and the alcohol injected from the well of the multi-well plate kit to the pipette are held in the high-temperature reaction tube, flowing in or out air by heating the high-temperature reaction block or by an upward or downward movement of the piston or both. Before the biological sample is mixed with the coupling solution contained in a unit well of the multi-well plate kit, the biological sample mixed with the cell lysis solution may be injected to the high-temperature reaction tube using the pipette so as to allow easy cell lysis of the biological sample.

### ADVANTAGEOUS EFFECTS

Using the apparatus of the present invention for separating target materials from a plurality of biological samples by using magnetic particles to which the magnetic particles are to be reversibly coupled, wherein a plurality of pipettes arranged in at least two rows are used to treat a plurality of biological samples, twice the number of samples can be fully automatically treated as compared to the existing apparatus having pipettes arranged in only one row.

The present invention is advantageous in that sample and reagent loading can be done simply and quickly using a multi-well plate such as a 96-well plate.

According to the present invention, samples of various volumes can be automatically refined quickly since magnetic particles may be separated from the samples and suspended using pipettes optimized for four different functions.

According to the present invention, the pipettes are mounted and released automatically and, after use, the contaminated pipettes are discarded to protect the user from contact with pathogens. Further, since a sterilizing device is provided inside the apparatus, pathogenic samples can be handled sanitarily.

According to the present invention, when a biological sample or a clinical sample is treated, holes are made minimally on a film attached on a 96-well plate for injection of the sample. Hence, contact with air and contamination of the sample resulting therefrom may be prevented.

According to the present invention, magnets are approached close to both sides of a pipette by means of the magnet mounting unit in order to apply a stronger magnetic field to the pipette. Consequently, due to the magnetic field applied by the magnet mounting unit, only the magnetic particles with nucleic acids bound thereto are attached uniformly on the inner periphery of the pipette and are collected effectively. Therefore, the nucleic acids bound to the magnetic particles can be separated with high purity without loss.

If the alcohol remaining on the magnetic particles is eluted together with the nucleic acids during the elution using a nucleic acid eluent, direct or indirect reactions may occur with enzymes used for polymerase chain reaction, real-time polymerase chain reaction, sequencing reaction, etc. This may result in decreased activity and sensitivity of the enzymes. According to the present invention, the alcohol in the washing solution that may remain on the magnetic particles can be completely removed before the elution of the nucleic acids using the nucleic acid eluent.

### BRIEF DESCRIPTION OF THE DRAWINGS

FIG. 1 and FIG. 2 schematically show major parts of an apparatus according to Embodiment 1.

FIG. 3 schematically shows the apparatus according to Embodiment 1 with a casing partly removed.

FIG. 4 is a perspective view of a base plate of the apparatus according to Embodiment 1.

FIG. 5 illustrates a use of the base plate of the apparatus according to Embodiment 1.

FIG. 6 illustrates a loading of a high-temperature reaction tube shown in FIG. 5.

FIG. 7 illustrates an accommodation of the base plate of the apparatus according to Embodiment 1 in the casing.

FIG. 8 is a perspective view of a multi-well plate kit according to Embodiment 1.

FIG. 9 illustrates an extraction of DNA from blood according to Embodiment 1.

FIG. 10 illustrates a polymerase chain reaction using the DNA extracted from blood according to Embodiment 1.

FIGS. 11 to 13 schematically show major parts of an apparatus according to Embodiment 2.

FIG. 14 is a flowchart according to Embodiment 4.

FIG. 15 is a graph showing a result of performing real-time polymerase chain reaction with different concentrations of ethyl alcohol.

FIG. 16 is a graph showing a result of performing real-time polymerase chain reaction using DNAs extracted according to Embodiment (#1, #2, #3).

### DESCRIPTION OF REFERENCE NUMERALS OF THE MAIN ELEMENTS IN THE DRAWINGS

| | |
|---|---|
| 100: pipette block | 200: fixing body |
| 300: casing | 400: base plate |

### MODE FOR CARRYING OUT THE INVENTION

Hereinafter, embodiments of the present invention will be described in detail referring to the attached drawings.

### Embodiment 1

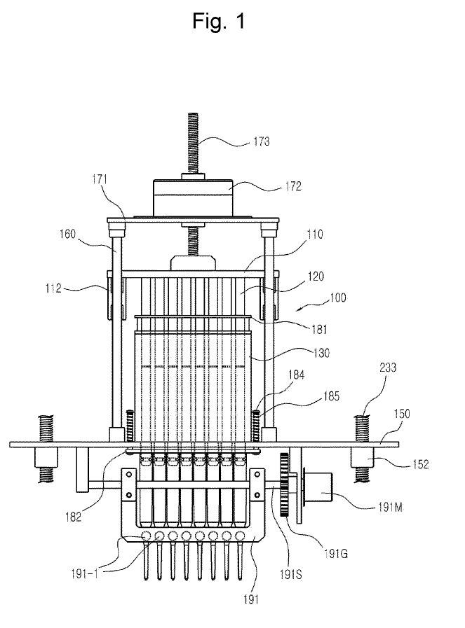

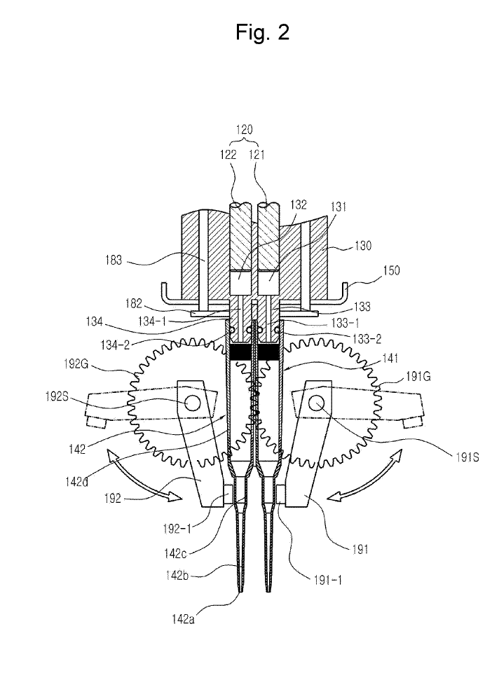

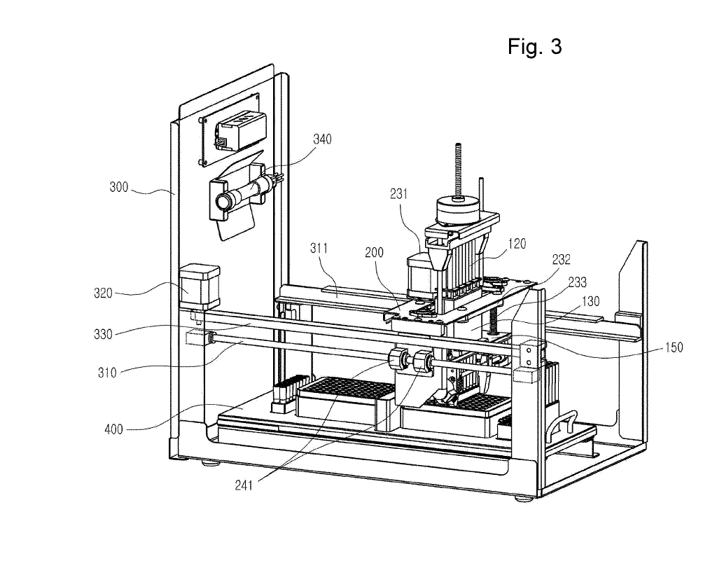

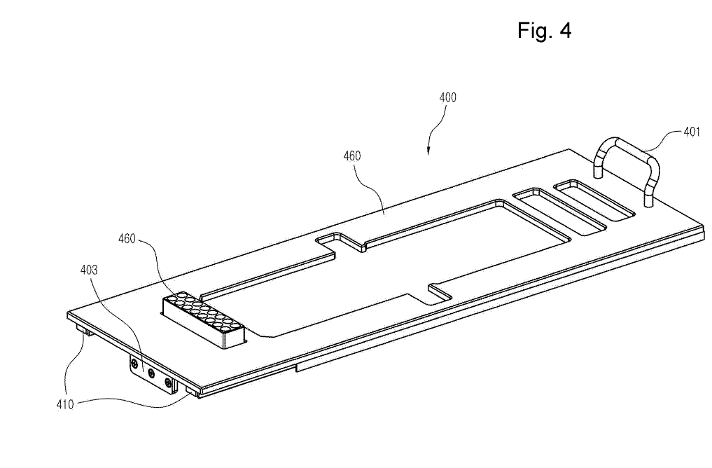

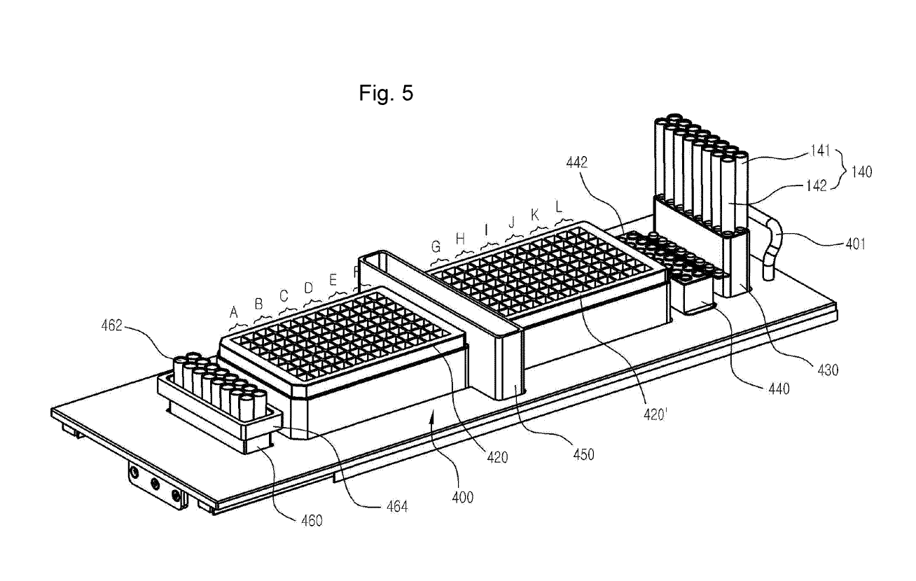

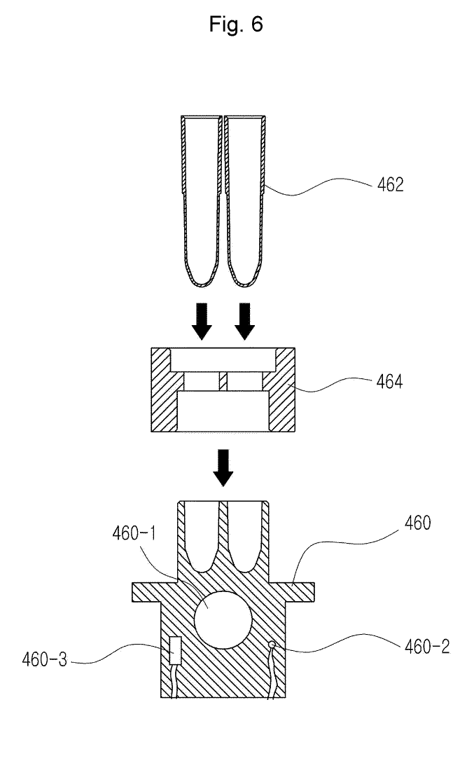

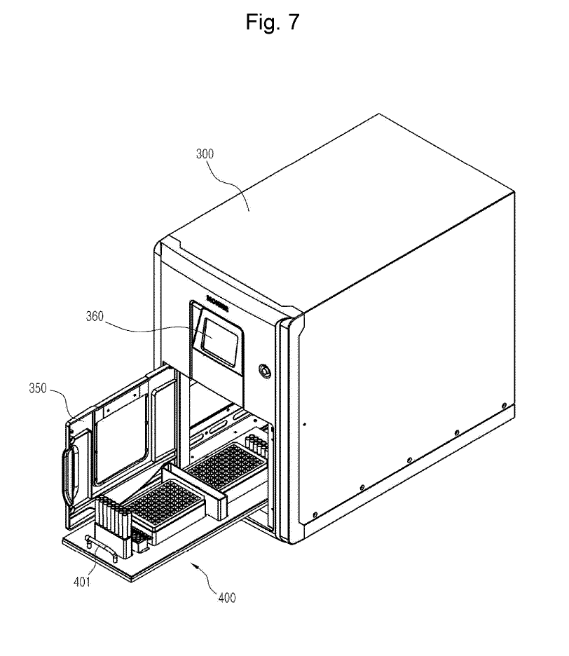

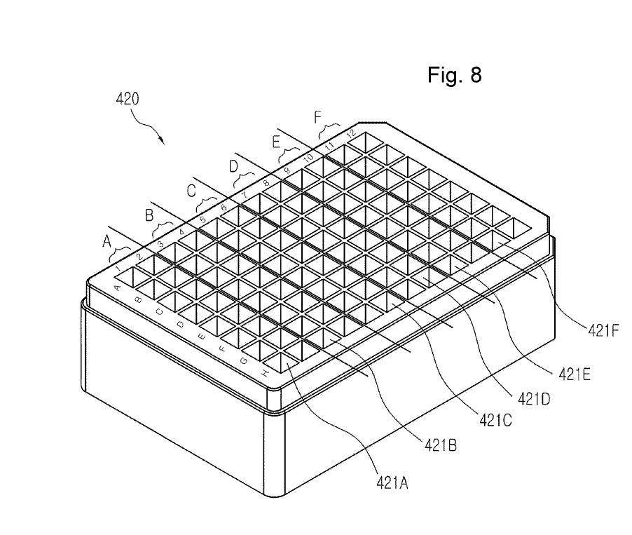

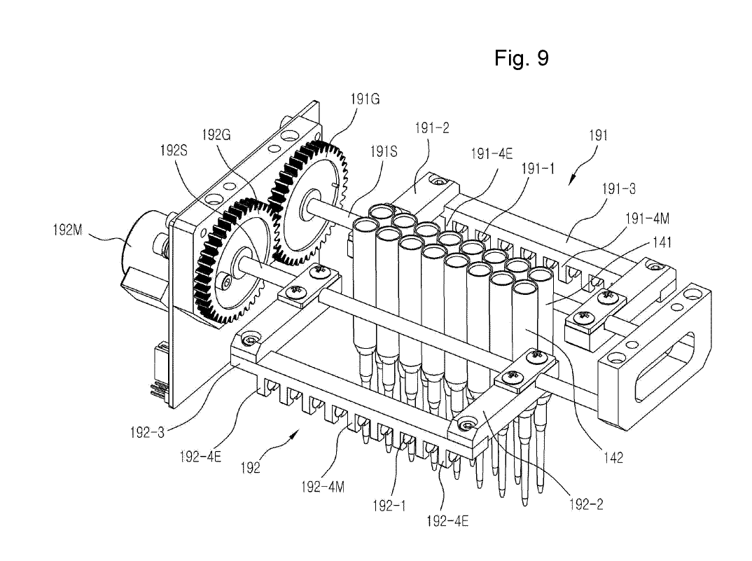

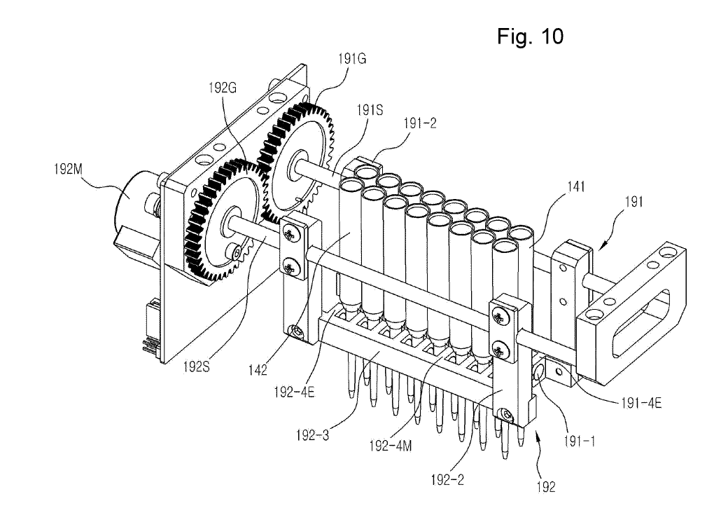

Embodiment 1 relates an automatic refining apparatus of the present invention, i.e. an apparatus for separating target materials from a plurality of biological samples by using magnetic particles to which the magnetic particles are to be reversibly coupled. FIG. 1 and FIG. 2 schematically show major parts of an apparatus according to Embodiment 1. FIG. 3 schematically shows the apparatus according to Embodiment 1 with a casing partly removed. FIG. 4 is a perspective view of a base plate of the apparatus according to Embodiment 1. FIG. 5 illustrates a use of the base plate of the apparatus according to Embodiment 1. FIG. 6 illustrates a loading of a high-temperature reaction tube shown in FIG. 5. FIG. 7 illustrates an accommodation of the base plate of the apparatus according to Embodiment 1 in the casing. FIG. 8 is a perspective view of a multi-well plate kit according to Embodiment 1. FIG. 9 illustrates an extraction of DNA from blood according to Embodiment 1. FIG. 10 illustrates a polymerase chain reaction using the DNA extracted from blood according to Embodiment 1.

An apparatus according to Embodiment 1 comprises a pipette block 100, a fixing body 200, a magnetic field application unit, a pipette block upward/downward moving means, a pipette block forward/backward moving means, a casing 300 and a base plate 400.

Referring to FIG. 1, the pipette block 100 has a piston fixing plate 110. Referring to FIGS. 2 and 3, a plurality of pistons 120 are provided in two rows on a lower surface of the piston fixing plate 110. The plurality of pistons 120 comprises first row pistons 121 (see FIG. 2) and second row pistons 122 (see FIG. 3) of the same number as the first row pistons 121. For example, the number of the first row pistons 121 and the second row pistons 122 may be 8 or 12.

Referring to FIGS. 1 to 3, the pipette block 100 has a piston guiding unit 130. The piston guiding unit 130 has piston guide holes 131, 132 guiding the upward and downward movement of the plurality of pistons 120. The piston guide holes 131, 132 may be formed from the upper end of the piston guiding unit 130 to near the lower end.

Referring to FIG. 2, pipette mounting units 133, 134 are provided in two rows below the piston guiding unit 130. The pipette mounting units 133, 134 have piston guide holes 131, 132 communicating with the connecting holes 133-1, 134-1. The connecting holes 133-1, 134-1 re formed from a lower end of the pipette mounting units 133, 13 to an upper portion thereof. As the piston guiding unit 130 moves downward, the pipette mounting units 133, 134 are engaged with an upper end of an inner periphery of a plurality of pipettes 141, 142 arranged in two lows below the pipette mounting units 133, 134. An outer periphery of the pipette mounting units 133, 134 may be engaged with engagement rings 133-2, 134-2. Asa result, the pipette mounting units 133, 134 may be engaged with the upper end of the inner periphery of pipettes 141, 142. The pipette mounting units 133, 134 are formed with the same shape such that, when a plurality of pipettes 141, 142 are mounted, the heights of the pipettes are identical. Accordingly, as will be described later, the same magnetic field can be applied to the plurality of pipettes 141, 142 by a magnetic field application unit.

Referring to FIGS. 1 and 2, the lower end of the piston guiding unit 130 is supported and fixed by a piston guiding unit supporting plate 150. Referring to FIG. 2, the piston guiding unit supporting plate 150 has through-holes allowing the pipette mounting units 133, 134 to penetrate therethrough.

Referring to FIG. 1, a fixing nut 152 is provided at the piston guiding unit supporting plate 150 of the pipette block 100. The fixing nut 152 is engaged with an up/down movement screw 233 such that rotation relative to each other is allowed.

Referring to FIG. 3, an upper end of the up/down movement screw 233 is connected to the fixing body 200, such that rotation relative to the fixing body 200 is possible but upward and downward movement is impossible. Referring to FIG. 3, an up/down movement motor 231 is provided at the fixing body 200 and an up/down moving belt 232 is connected to the up/down movement motor 231. As the up/down moving belt 232 is moved, the up/down movement screw 233 rotates, and the piston guiding unit supporting plate 150 moves up and down with respect to the fixing body 200. The up/down moving belt 232 may be a timing belt.

Referring to FIG. 1, the pipette block 100 has a guide rod 160. The guide rod 160 protrudes from an upper surface of the piston guiding unit supporting plate 150. The guide rod 160 is engaged with the piston fixing plate 110 and guides the upward and downward movement of the piston fixing plate 110. A guide member 112 for guiding the upward and downward movement of the piston fixing plate 110 may be fixedly connected with the piston fixing plate 110.

Referring to FIG. 1, a piston driving motor supporting plate 171 is provided at the upper end of the guide rod 160. A piston driving motor 172 is mounted at the piston driving motor supporting plate 171. A piston control screw 173 is connected to the piston driving motor 172 so as to allow upward and downward movement as it rotates. A lower end of the piston control screw 173 is connected to the piston fixing plate 110 such that upward and downward movement is impossible although rotation relative thereto is allowed.

Referring to FIG. 1, a detachable upper plate 181 is provided above the piston guiding unit 130. The detachable upper plate 181 has through-holes so that the plurality of pistons 120 penetrate therethrough.

Referring to FIG. 2, a detachable lower plate 182 is provided below the piston guiding unit supporting plate 150. The detachable lower plate 182 has through-holes so that the plurality of pipette mounting units 133, 134 pass therethrough. The through-holes through which the pipette mounting units 133, 134 penetrate are formed to have such a size that the penetration of the plurality of pipette mounting units 133, 134 is allowed but the penetration of the plurality of pipettes 141, 142 mounted on the pipette mounting units is not allowed. Accordingly, as the detachable lower plate 182 moves downward, it presses down the upper end of the plurality of pipettes 141, 142 mounted on the pipette mounting units and separates the plurality of pipettes 141, 142 therefrom. The detachable upper plate 181 and the detachable lower plate 182 are connected by a connecting rod 183 with a gap. In order to install the connecting rod 183, through-holes are formed on the piston guiding unit 130.

Referring to FIG. 1, a protruding rod 184 is provided on an upper surface of the detachable lower plate 182. The protruding rod 184 protrudes above the piston guiding unit supporting plate 150 via through-holes (not shown in the figure) formed on the piston guiding unit supporting plate 150. The protruding rod 184 is inserted in a spring 185. A lower end of the spring 185 is elastically supported by the upper surface of the piston guiding unit supporting plate 150 and an upper end thereof is elastically supported by an upper end of the protruding rod 184. Accordingly, it exerts an elastic force so that the detachable lower plate 182 is fastened to the piston guiding unit supporting plate 150. Also referring to FIG. 2, when the piston fixing plate 110 moves downward and presses the detachable upper plate 181, if the pressing force is greater than the elastic force of the spring 185, the detachable lower plate 182 is moved downward so as to separate the plurality of pipettes 141, 142.

That is to say, as the pipette mounting units 133, 134 move downward, the plurality of pipettes 141, 142 are mounted on the pipette mounting units 133, 134, and, as the detachable lower plate 182 moves downward, the mounted plurality of pipettes 141, 142 are separated from the pipette mounting units 133, 134. Also, as the pistons 120 move upward and downward, biological samples including target materials are sucked into or discharged out of the plurality of pipettes 141, 142.

Referring to FIG. 2, the plurality of pipettes 141, 142 mounted on the pipette mounting units 133, 134 are configured to serve four major functions. Since the pipette 141 and the pipette 142 are identical, description will be given only about the pipette 142. A tip 142a at a lower end of the pipette 142 is formed to be pointed so that a hole may be made easily on a film (not shown in the figure) of a multi-well plate kit 420, 420′, as will be described later. A solution passage 142b is formed to be as thin as possible, so that it may reach bottom portions of wells 421A, 421B, 421C, 421D, 421E, 421F of the multi-well plate kit 420 and the volume of a solution retained therein may be minimized. A magnetic particle collecting unit 142c is configured such that, when a magnet is approached from outside, magnetic particles contained in a liquid flowing downward may be attached to an inner wall thereof by the magnetic force. If the magnetic particle collecting unit 142c has a large inner diameter, the magnetic particles at the opposite side of the magnet may flow downward without being attached to the inner wall. Thus, the magnetic particle collecting unit 142c is formed to have such a radius that the magnetic particles passing through the opposite side of the magnet may also be collected. A solution storage unit 142d is formed to have an inner diameter and a length such that as large a volume as possible may be contained therein within the distance 9 mm between neighboring wells of the 96-well plate kit.

Referring to FIGS. 1 and 2, the magnetic field application unit applies and releases a magnetic field to and from the pipettes 141, 142 mounted in the pipette block 100. The magnetic field application unit comprises a first row magnet mounting unit 191, a magnet mounting unit motor 191M, a first row gear 191G, a first row rotation shaft 191S, a second row magnet mounting unit 192, a second row gear 192G and a second row rotation shaft 192S.

Referring to FIGS. 1 and 2, magnets 191-1 for applying a magnetic field to the first row pipettes 141 mounted on the first row pipette mounting unit 133 are provided at the first row magnet mounting unit 191. In particular, referring to FIG. 1, the number of the magnets 191-1 may be the same as that of the first row pipettes 141.

Referring to FIGS. 1 and 2, the first row gear 191G is connected to the piston guiding unit supporting plate 150 and rotated by the magnet mounting unit motor 191M. The first row rotation shaft 191S is connected to the first row gear 191G and is rotated as the first row gear 191G rotates. The first row magnet mounting unit 191 is radially connected to the first row rotation shaft 191S such that the distance between the magnets 191-1 and the first row pipettes 141 changes as the first row rotation shaft 191S rotates. As the distance between the magnets 191-1 and the first row pipettes 141 increases, the magnetic field applied to the first row pipettes 141 is released. Accordingly, the magnet mounting unit motor 191M, the first row gear 191G and the first row rotation shaft 191S serve as a moving means to move the first row magnet mounting unit 191.

Referring to FIG. 2, magnets 192-1 for applying a magnetic field to the second row pipettes 142 mounted on the second row pipette mounting unit 134 are provided at the second row magnet mounting unit 192. Although not shown in the figure, the number of the magnets 192-1 may be the same as that of the second row pipettes 142.

Referring to FIG. 2, the second row gear 192G is engaged with the first row gear 191G and is rotated as the first row gear 191G rotates. The second row rotation shaft 192S is connected to the second row gear 192G and is rotated as the second row gear 192G rotates. The second row magnet mounting unit 192 is radially connected to the second row rotation shaft 192S such that the distance between the magnets 192-1 and the second row pipettes 142 changes as the second row rotation shaft 192S rotates. As the distance between the magnets 192-1 and the second row pipettes 142 increases, the magnetic field applied to the second row pipettes 142 is released. Accordingly, the second row gear 192G and the second row rotation shaft 192S serve as a moving means to move the second row magnet mounting unit 192.

In Embodiment 1, the magnitude and duration of the magnetic field applied to each of the first row pipettes 141 by the first row magnet mounting unit 191, the first row gear 191G and the first row rotation shaft 191S are the same as the magnitude and duration of the magnetic field applied to each of the second row pipettes 142 by the second row magnet mounting unit 192, the second row gear 192G and the second row rotation shaft 192S. Accordingly, the first row gear 191G is the same as the second row gear 192G, the first row rotation shaft 191S is symmetrical to the second row rotation shaft 192S, and the first row magnet mounting unit 191 is the same as the second row magnet mounting unit 192. Therefore, the first row magnet mounting unit 191 and the second row magnet mounting unit 192 that create the same magnetic field rotate symmetrically to each other. The magnets 191-1, 192-1 may be disc-shaped permanent magnets, preferably super strong magnets made from neodymium, samarium/cobalt, alnico, or the like.

In Embodiment 1, the second row gear 192G, rather than the first row gear 191G, may be driven by the magnet mounting unit motor 191M.

Referring to FIGS. 3 and 7, a forward/backward movement supporting rod 310 is provided at the fixing body 200 along a forward/backward direction.

Referring to FIG. 3, a forward/backward movement slider 241 is provided at the forward/backward movement supporting rod 310. The forward/backward movement slider 241 is fixed to the fixing body 200. A forward/backward movement motor 241 is provided at the casing 300. A forward/backward moving belt 330 is connected to the forward/backward movement motor 241. A portion of the moving belt 330 is attached to the fixing body 200. Thus, as the moving belt 330 moves, the fixing body 200 is moved forward and backward along the forward/backward movement supporting rod 310.

Referring to FIG. 3, a forward/backward guider 311 is provided at the opposite side of the forward/backward movement supporting rod 310 in order to support the other side of the fixing body 200 and guide the forward/backward movement thereof.

Referring to FIG. 3, the base plate 400 is located below the fixing body 200. Referring to FIG. 4, a sliding rail 410 may be provided at a lower end of the base plate 400 so as to allow sliding movement of the casing 300.

Referring to FIGS. 4 and 5, on the base plate 400, the multi-well plate kits 420, 420′, a pipette rack 430 insertably holding the plurality of pipettes 140 mounted on the pipette block 100 in two rows, a sample storage tube rack 440 insertably holding a plurality of sample storage tubes 442 for storing the purified sample in two rows, and a waste bottle 450 for holding waste solution discarded from the plurality of pipettes 140 mounted on the pipette block 100 are provided. The base plate 400 may have a high-temperature reaction block 460 for heating a plurality of high-temperature reaction tubes 462 insertably held in two rows mounted thereon. Referring to FIG. 6, the high-temperature reaction tubes 462 may be insertably mounted on the high-temperature reaction block 460 via a high-temperature reaction tube rack 464. The high-temperature reaction tube rack 464 may be made of a plastic material with low thermal conductivity so as to allow the user to hold it in hands. Reference numeral 460-1 is a heater, reference numeral 460-2 is a power supply unit, and reference numeral 460-3 is a heat blocking unit to maintain a constant temperature.

Referring to FIG. 3, a sterilizing device such as a UV lamp 340 or an ozone generator (not shown in the figure) may be provided in the casing 300.

FIG. 8 shows a multi-well plate kit 420 which is accommodated in the casing 300 being mounted on the base plate 400 and located below the pipette block 100.

Referring to FIG. 8, the multi-well plate kit 420 comprises a plurality of unit wells A, B, C, D, E, F arranged in two neighboring rows and a film (not shown in the figure) sealing an upper end of the plurality of unit wells A, B, C, D, E, F. The multi-well plate kit 420 may be a 96-well plate kit. Differently from FIG. 8, the multi-well plate kit 420 may comprise unit wells arranged in one row. The unit well A may be sealed after injecting protease, RNase or sample pretreatment buffer thereto. The unit well B may be sealed after injecting a cell lysis solution for lysing the biological sample thereto. The unit well C may be sealed after injecting a coupling solution thereto. The unit well D may be sealed after injecting an aqueous suspension of magnetic particles thereto. The unit well E may be sealed after injecting a washing solution thereto. The unit well F may be sealed after injecting an eluent thereto. That is to say, the solutions for separation of the target materials are contained in the unit wells excluding at least one unit well (s) such that the same solution is contained in the same unit well.

If the solution contained in one of the sealed unit wells is an aqueous suspension of magnetic particles, the magnetic particles suspended in the aqueous suspension may be spherical magnetic particles coated with silica.

Hereinafter, operation of the apparatus according to Embodiment 1 will be described.

Referring to FIGS. 4 and 5, the multi-well plate kits 420, 420′, e.g. 96-well plate kits, are mounted on the upper surface of the base plate 400 via holes formed thereon. The sliding rail 410 is provided at the lower side of the base plate 400, so that the base plate 400 may be pulled out of the casing 300 handle 401 to mount necessary items thereon, as shown in FIG. 7. Referring to FIGS. 4 and 5, in order to operate the apparatus of Embodiment 1, the well plate kit 420, 420′, the waste bottle 450, or the like are placed on the base plate 400. The preparation procedure will be described in detail. First, the number of the biological samples including the target materials has to be determined. The apparatus according to Embodiment 1 is capable of flexibly refining from 1 up to 16 samples. As a specific example, FIG. 5 shows a procedure of preparing 16 samples. The multi-well plate kit 420, which is a 96-well plate kit, holds magnetic particles as well as various solutions and serves as a plate for injecting and holding the biological samples. First, holes are made on the film sealing the unit well A of the 96-well plate kit using the pipette tip corresponding to the number of the biological samples, and then the biological samples are injected into each well 421A, one by one. Then, the 96-well plate kit is mounted on the base plate 400 and then another 96-well plate kit holding other solutions is mounted on the base plate 400. In addition, the waste bottle 450 for collecting waste solution produced during the refining process is mounted. If high-temperature reaction is required, reaction is carried out on the high-temperature reaction block 460. After a required number of the high-temperature reaction tubes 462 are mounted on the high-temperature reaction tube rack 464, as illustrated in FIG. 6, the rack is inserted into the high-temperature reaction block 460. If high-temperature reaction is not necessary, mounting of the high-temperature reaction tubes 462 and the high-temperature reaction tube rack 464 is unnecessary. Then, a plurality of pipettes 140 are mounted using the pipette rack 430 such that the locations of the pipettes 140 correspond to those of the samples, as shown in FIG. 5. Also, the same number of sample storage tube 442 are mounted using the sample storage tube rack 440. The sample storage tube 442 may be a standard product for use in a 96-well plate kit such as the 8-strip tube for PCR (In FIG. 5, a total of 16 samples are mounted on all the wells). If less than 16 wells are used, it is important to match the locations of the pipettes 140 with those of the sample storage tubes 442 and the high-temperature reaction tubes 462. For this, it is desired to mount the respective pipettes 140, sample storage tubes 442 and high-temperature reaction tubes 462 after placing their racks, i.e. the pipette rack 430, the sample storage tube rack 440 and the high-temperature reaction tube rack 464, in parallel.

After the mounting is completed, the base plate 400 is pushed until it no longer moves because of a stopper 403. Then, a door 350 of the casing 300 is closed and then automatic refining is performed by manipulating a touch screen 360. After the automatic refining is completed in about 30 minutes, the door 350 is opened and the base plate 400 is pulled out. Then, the refined samples are recovered from the sample storage tube rack 440 holding the purified nucleic acids. Then, after taking out the used pipettes and the high-temperature reaction tube rack 464, lids of the sample storage tubes 442 are closed. The sample storage tubes 442 may be directly subjected to the necessary experiments or may be stored in a refrigerator of −20° C. All of the 96-well plate kits, pipettes, waste bottle, etc. used for the nucleic acid extraction from the base plate 400 and discarded. After pushing the base plate 400 until it no longer moves because of the stopper 403 and closing the door, the inside of the apparatus is sterilized using the UV lamp 340. The 96-well plate kit is discarded if all of its 16 wells were used. It may be reused if there are unused wells.

Excluding the preparation process and post-treatment process, the remaining refining process may be carried out by an automatic device and computer circuitry of the automatic refining apparatus. For this procedure, the plurality of pipettes 140 arranged in tow rows are automatically mounted on the pipette block 100 by the pipette mounting units 133, 134.

The upward and downward movement of the pipette block 100 is aided by the up/down movement screw 233, and the forward and backward movement is aided by the forward/backward moving belt 330. The up/down movement screw 233 and the forward/backward moving belt 330 allow to perform works at desired locations.

### Test Example 1

### Extraction of Chromosomal DNA Using the Apparatus of Embodiment 1

Manufacture of Kit for Chromosomal DNA Extraction

In order to manufacture a kit for chromosomal DNA extraction, adequate amounts of previously prepared reagents were added to the unit wells B through E of the 96-well plate kit. Compositions of the reagents used for the chromosomal DNA extraction are as follows. In the unit well B of the 96-well plate kit, a cell lysis solution (pH 4.0-7.0) comprising 1-8 M guanidine hydrochloride, 10-100 mM tris hydrochloride, 10-500 mM sodium chloride and 1-50% surfactant (Triton X-100, Tween-20, Tween-80, NP-40, etc.) was added as a lysis buffer for lysing the cells of the biological samples. In the unit well C, alcohol (isopropyl alcohol or ethyl alcohol) was added to improve binding of the chromosomal DNA to the magnetic particles. In the unit well D, an aqueous suspension of magnetic particles was added. In the unit well E, a washing solution comprising 1-8 M guanidine hydrochloride, 10-100 mM tris hydrochloride, 10-500 mM sodium chloride and 10-90% alcohol (isopropyl alcohol or ethyl alcohol) was added to selectively remove impurities while retaining the binding of DNA to the magnetic particles. In the unit well F, a nucleic acid eluent (pH 8.0-9.0) comprising 1-50 mM tris hydrochloride was added to elute DNA from the magnetic particles so as to obtain pure DNAs.

2) Extraction of Chromosomal DNA from Whole Blood

Whole blood (200 μL) was placed in the unit well A of thus prepared DNA extraction kit. After mounting the DNA extraction kit, the waste bottle, the high-temperature reaction tube rack which combined with reaction tube, the pipette rack which combined with reaction tube and the sample storage tube rack which combined with reaction tube on their respective locations of the automatic refining apparatus, DNA extraction from the whole blood was carried out automatically by selecting a preset method.

The preset method for extraction of from the whole blood includes all the procedures required for DNA extraction, upward and downward movement of the pipettes, movement of the magnets for transferring the magnetic particles, movement of the pipettes for transferring the solutions held in the multi-well plates, kind and volume of the solutions held in the multi-well plates, location and volume of the waste solution to be discarded, location of the tubes and duration for the high-temperature reaction, sterilization using the UV lamp upon completion of the nucleic acid purification, or the like.

3) Identification of Extracted Chromosomal DNA

Yield, concentration and purity of the extracted chromosomal DNA were measured by UV absorption spectroscopy. First, after baseline measurement at 260 nm, 280 nm and 320 nm using sterilized triple distilled water, absorbance of the extracted DNA was measured at the respective wavelengths. From the absorbance measurement values, yield, concentration and purity were calculated according to the following equations.

Concentration of extracted DNA=(absorbance at 260 nm−absorbance at 320 nm)×50×dilution factor

Yield of extracted DNA=(concentration of extracted DNA)×(volume of eluent)

Purity of extracted DNA=(absorbance at 260 nm−absorbance at 320 nm)/(absorbance at 280 nm−absorbance at 320 nm)

The calculation result is shown in the following table. Average concentration of chromosomal DNA separated from the 16 samples was 36 ng/μL. Average yield was 3.6 ng and average purity was 1.95. A very good result was obtained.

| | Sample # | | | | | | | | |
|---|---|---|---|---|---|---|---|---|---|
| | 1 | 2 | 3 | 4 | 5 | 6 | 7 | 8 | 9 |
| O.D₂₆₀ | 0.072 | 0.069 | 0.075 | 0.077 | 0.074 | 0.078 | 0.075 | 0.074 | 0.077 |
| O.D₂₈₀ | 0.038 | 0.036 | 0.040 | 0.039 | 0.038 | 0.039 | 0.040 | 0.037 | 0.035 |
| O.D₃₂₀ | 0.001 | 0.000 | 0.003 | 0.000 | 0.002 | 0.000 | 0.000 | 0.002 | 0.001 |
| Dilution factor | 10 | 10 | 10 | 10 | 10 | 10 | 10 | 10 | 10 |
| Elution Vol. (ul) | 100 | 100 | 100 | 100 | 100 | 100 | 100 | 100 | 100 |
| Conc. (ng/ul) | 35.5 | 34.5 | 36 | 38.5 | 36 | 39 | 37.5 | 36 | 38 |
| Yield (ug) | 3.6 | 3.5 | 3.6 | 3.9 | 3.6 | 3.9 | 3.8 | 3.6 | 3.8 |
| Purity | 1.92 | 1.92 | 1.95 | 1.97 | 2.00 | 2.00 | 1.88 | 2.06 | 2.24 |

| | Sample # | | | | | | | |
|---|---|---|---|---|---|---|---|---|
| | 10 | 11 | 12 | 13 | 14 | 15 | 16 | Average |
| O.D₂₆₀ | 0.079 | 0.072 | 0.071 | 0.066 | 0.068 | 0.072 | 0.069 | |
| O.D₂₈₀ | 0.041 | 0.038 | 0.037 | 0.037 | 0.036 | 0.039 | 0.038 | |
| O.D₃₂₀ | 0.003 | 0.000 | 0.000 | 0.001 | 0.001 | 0.001 | 0.002 | |
| Dilution factor | 10 | 10 | 10 | 10 | 10 | 10 | 10 | |
| Elution Vol. (ul) | 100 | 100 | 100 | 100 | 100 | 100 | 100 | |
| Conc. (ng/ul) | 38 | 36 | 35.5 | 32.5 | 33.5 | 35.5 | 33.5 | 35.97 |
| Yield (ug) | 3.8 | 3.6 | 3.6 | 3.3 | 3.4 | 3.8 | 3.4 | 3.6 |
| Purity | 2.00 | 1.89 | 1.92 | 1.81 | 1.91 | 1.87 | 1.86 | 1.95 |

The extracted DNA (100 ng) was subjected to electrophoresis on 1% agarose gel. In FIG. 9, lane M is for a Bioneer's size marker (Cat. No. D-1040) and lanes 1 to 16 are for the extracted DNA. As seen from the figure, no decomposition or inclusion of impurities (e.g. RNA) occurred during the procedure of extracting chromosomal DNA from the whole blood.

Further, the extracted DNA (10 ng) was subjected to GAPDH gene amplification using a PCR primer capable of amplifying the gene and Bioneer's AccuPower PCR premix under the following conditions: 1 minute at 94° C. for DNA denaturation; 1 minute at 60° C. for attachment of each primer at the target site; and 40 cycles of 3 minutes at 72° C. for preparation of double stranded DNA. Following the polymerase chain reaction, the PCRproduct (5 μL) was subjected to electrophoresis on 1% agarose gel to identify the size of the PCR product. It was demonstrated that the extracted DNA can be used in other experiments. In FIG. 10, lane M is for a Bioneer's size marker (Cat. No. D-1070) and lanes 1 to 16 are for the PCR products. All the PCR products had exactly the same size.

### Embodiment 2

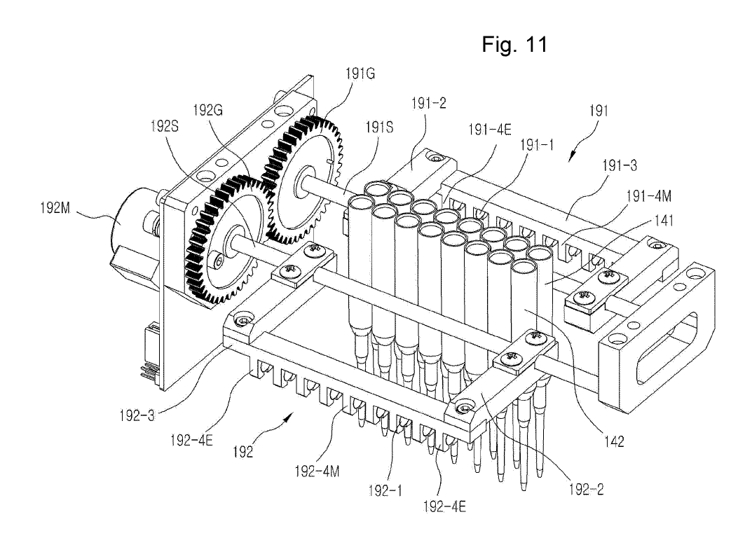

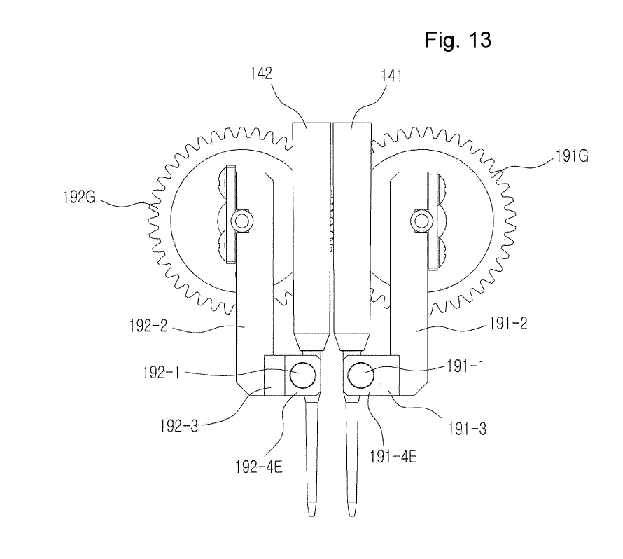

Embodiment 2 relates to another automatic refining apparatus of the present invention, i.e. an apparatus for separating target materials from a plurality of biological samples by using magnetic particles to which the magnetic particles are to be reversibly coupled. FIGS. 11 to 13 schematically show major parts of an apparatus according to Embodiment 2.

Referring to FIGS. 11 to 13, a magnetic field application unit comprises a first row magnet mounting unit 191, a first row gear 191G, a first row rotation shaft 191S, a second row magnet mounting unit 192, a magnet mounting unit motor 192M, a second row gear 192G and a second row rotation shaft 192S.

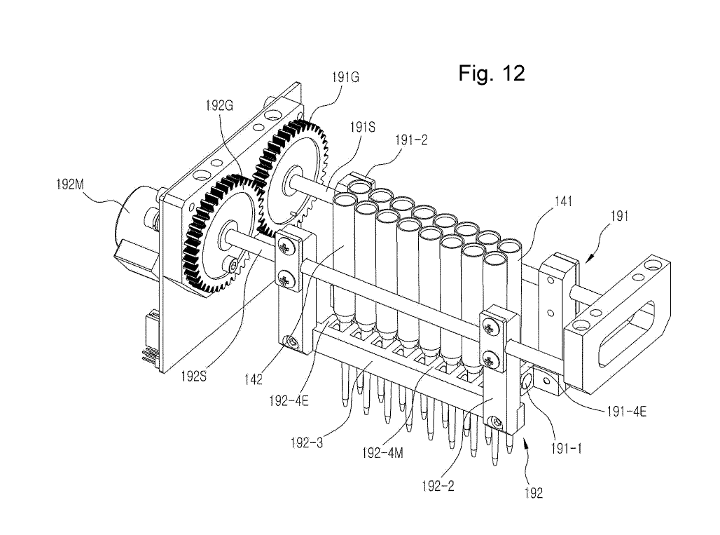

Referring to FIGS. 11 and 12, the second row magnet mounting unit 192 comprises a second row rotating arm 192-2 and a second row plate mount 192-3.

Referring to FIGS. 11 and 12, the second row rotating arm 192-2 is radially connected and fixed to the second row rotation shaft 192S. The second row plate mount 192-3 is fixed at the end portion of the second row rotating arm 192-2 so as to be in parallel with the second row rotation shaft 192S.

Referring to FIGS. 11 and 12, a second row middle plate 192-4M and a second row end plate 192-4E are provided on the second row plate mount 192-3 with the same interval. As the second row rotation shaft 192S rotates, the second row middle plate 192-4M is located between neighboring pipettes among second row pipettes 142. On the second row middle plate 192-4M, through-holes are formed along a direction parallel to the row direction of the second row pipettes 142 so that magnets 192-1 may be mounted. As the second row rotation shaft 192S rotates, the second row endplate 192-4E is located outside of a pipette located at the side end among the second row pipettes 142. On the second row endplate 192-4E, through-holes are formed along a direction parallel to the row direction of the second row pipettes 142 so that magnets 192-1 may be mounted. The through-holes formed on the second row middle plate 192-4M and the through-holes formed on the second row end plate 192-4E are in one line.

Referring to FIGS. 11 and 12, the second row gear 192G is rotated by the magnet mounting unit motor 192M. The second row rotation shaft 192S is connected to the second row gear 192G and is rotated as the second row gear 192G rotates. The second row magnet mounting unit 192 is radially connected to the second row rotation shaft 192S such that the distance between the magnets 192-1 and the second row pipettes 142 changes as the second row rotation shaft 192S rotates. As the distance between the magnets 192-1 and the second row pipettes 142 increases, the magnetic field applied to the second row pipettes 142 is released. Accordingly, the magnet mounting unit motor 192M, the second row gear 192G and the second row rotation shaft 192S serve as a moving means to move the second row magnet mounting unit 192.

Referring to FIGS. 11 and 12, the first row magnet mounting unit 191 comprises a first row rotating arm 191-2 and a first row plate mount 191-3.

Referring to FIGS. 11 and 12, the first row rotating arm 191-2 is radially connected and fixed to the first row rotation shaft 191S. The first row plate mount 191-3 is fixed at the end portion of the first row rotating arm 191-2 so as to be in parallel with the first row rotation shaft 191S.

Referring to FIGS. 11 and 12, a first row middle plate 191-4M and a first row end plate 191-4E are provided on the first row plate mount 191-3 with the same interval. As the first row rotation shaft 191S rotates, the first row middle plate 191-4M is located between neighboring pipettes among first row pipettes 141. On the first row middle plate 191-4M, through-holes are formed along a direction parallel to the row direction of the first row pipettes 141 so that magnets 191-1 may be mounted. As the first row rotation shaft 191S rotates, the first row endplate 191-4E is located outside of a pipette located at the side end among the first row pipettes 141. On the first row end plate 191-4E, through-holes are formed along a direction parallel to the row direction of the first row pipettes 141 so that magnets 191-1 may be mounted. The through-holes formed on the first row middle plate 191-4M and the through-holes formed on the first row end plate 191-4E are in one line.

Referring to FIGS. 11 and 12, the first row gear 191G is engaged with the second row gear 192G and is rotated as the second row gear 192G rotates. The first row rotation shaft 191S is connected to the first row gear 191G and is rotated as the first row gear 191G rotates. The first row magnet mounting unit 191 is radially connected to the first row rotation shaft 1915 such that the distance between the magnets 191-1 and the first row pipettes 141 changes as the first row rotation shaft 191S rotates. As the distance between the magnets 191-1 and the first row pipettes 141 increases, the magnetic field applied to the first row pipettes 141 is released. Accordingly, the first row gear 191G and the first row rotation shaft 191S serve as a moving means to move the first row magnet mounting unit 191.

In Embodiment 2, the first row gear 191G, rather than the second row gear 192G, may be driven by the magnet mounting unit motor 192M.

Referring to FIG. 12, since the magnets are located on both sides of the pipettes 141, 142, the magnetic particles with the nucleic acids bound thereto can be effectively collected in the pipettes 141, 142 without loss. If the magnet is located only on one side of the pipettes 141, 142, the magnetic particles with the nucleic acids bound thereto may be attached and collected only one inner side of the pipettes 141, 142. In that case, the aggregated magnetic particles may be lost in the subsequent procedures, for example when the mixture held in the pipettes 141, 142, excluding the magnetic particles with the nucleic acids bound thereto, is discharged to a waste bottle 450 using the magnetic field application unit and pistons 120.

Referring to FIG. 12, as the magnets 191-1, 192-1 are approached to the pipettes 141, 142 using the magnet mounting units 191, 192, the strength of the magnetic field applied to the pipettes 141, 142 is significantly increased. Accordingly, as the magnetic field is applied using the magnet mounting units 191, 192, the magnetic particles with nucleic acids bound thereto are attached uniformly on the inner wall of the pipettes 141, 142 and are collected effectively. Therefore, the nucleic acids bound to the magnetic particles can be separated with high purity and high yield without loss. In Embodiment 2, the magnets 191-1, 192-1 are located by the magnet mounting units 191, 192 with the first row pipettes 141 and the second row pipettes 142 therebetween in order to apply the magnetic field thereto.

Others are the same as in Embodiment 1.

### Embodiment 3

Embodiment 3 relates to a multi-well plate kit for use in the automatic refining apparatus according to Embodiment 1 or Embodiment 2. A description thereof will be omitted since it is the same as that given with respect to Embodiment 1.

### Embodiment 4

Embodiment 4 relates to a method for extracting nucleic acids from biological samples using the automatic refining apparatus according to Embodiment 1 or Embodiment 2.

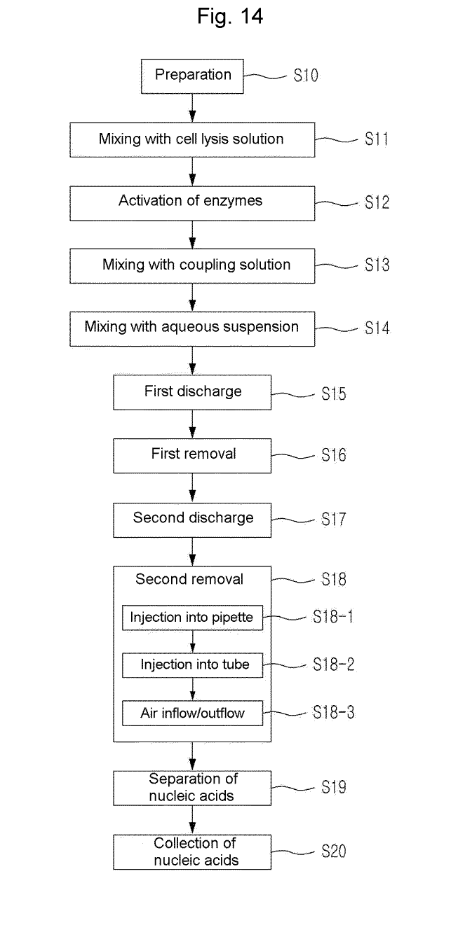

FIG. 14 is a flowchart according to Embodiment 4.

Referring to FIG. 14, Embodiment 4 comprises a preparation step (S10).

Referring to FIG. 5, in the preparation step (S10), two multi-well plate kits 420, 420′, a pipette rack 430 holding a plurality of pipettes 140 to be mounted on a pipette block 100 arranged in two rows, a sample storage tube rack 440 holding a plurality of sample storage tubes 442 for storing purified samples in two rows, a waste bottle 450 for holding a waste solution discarded from the plurality of pipettes 140, and a high-temperature reaction block 460 for heating a plurality of high-temperature reaction tubes 462 arranged in two rows are mounted on a base plate 400. Referring to FIG. 7, the base plate 400 is accommodated in a casing 300.

Referring to FIG. 14, Embodiment 4 comprises a step (S11) for mixing with a cell lysis solution.

Referring to FIG. 8, in the mixing step (S11), a biological sample held in a unit well A of the multi-well plate kit 420 is mixed with a cell lysis solution held in a unit well B of the multi-well plate kit 420, using pipettes 141, 142 (see FIG. 2).

Referring to FIG. 14, Embodiment 4 comprises an enzyme activation step (S12).

Referring to FIG. 5, in the enzyme activation step (S12), the biological sample mixed with the cell lysis solution is injected to the high-temperature reaction tube 462 using pipettes 141, 142 in order to facilitate cell lysis of the biological sample. Depending on the biological sample held in the unit well A of the multi-well plate kit 420 (see FIG. 8), the unit well A may be sealed after injecting an enzyme for cell lysis and protein degradation thereto. The enzymatic reaction is activated by the high-temperature reaction tube 462 and, as a result, the cells of the biological sample are completely lysed in short time.

Referring to FIG. 14, Embodiment 4 comprises a step (S13) for mixing with a coupling solution.

Referring to FIG. 8, in the step (S13) for mixing with a coupling solution, the cell lysis solution and the biological sample with the cells lysed are mixed with a coupling solution held in a unit well C of the multi-well plate kit 420, using the pipettes 141, 142. That is to say, in the step (S13) for mixing with a coupling solution, the mixture held in the high-temperature reaction tube 462 is injected into the unit well C of the multi-well plate kit 420. The coupling solution may be alcohol (isopropyl alcohol or ethyl alcohol) for improving the binding between nucleic acids and magnetic particles.

Referring to FIG. 14, Embodiment 4 comprises a step (S14) for mixing with an aqueous suspension.

Referring to FIG. 8, in the step (S14) for mixing with an aqueous suspension, the mixture with the coupling solution is mixed with an aqueous suspension of magnetic particles held in a unit well D of the multi-well plate kit 420, using the pipettes 141, 142. As a result thereof, target nucleic acids are attached on the surface of the magnetic particles.

Referring to FIG. 14, Embodiment 4 comprises a first discharge step (S15).

Referring to FIG. 8, in the first discharge step (S15), the mixture with the coupling solution is injected into the pipette 141, 142, and is located above the waste bottle 450. Subsequently, referring to FIG. 2, as the piston 120 moves downward, a first discharge pressure is applied to the pipette 141, 142 so that the mixture with the coupling solution is discharged from the pipette 141, 142 to the waste bottle 450. At the same time, a magnetic field is applied to the pipette 141, 142 by the magnet mounting units 191, 192 so that the magnetic particles and the materials attached to the magnetic particles are not discharged from the pipette 141, 142 but remain in the pipette 141, 142. Accordingly, in the first discharge step (S15), the mixture with the coupling solution excluding the magnetic particles and the materials attached thereto is discharged to the waste bottle 450.

Referring to FIG. 14, Embodiment 4 comprises a first removal step (S16).

Referring to FIG. 5, in the first removal step (S16), the magnetic field is released and the magnetic particles and the materials attached thereto are mixed with a washing solution held in a unit well E of the multi-well plate kit 420 using the pipette 141, 142 so as to wash them in the high-temperature reaction tube 462 or one of unit wells H, I, J, K and L of the multi-well plate kit 420 once or several times. The washing solution is one to selectively remove impurities attached to the magnetic particles while retaining the binding between the magnetic particles and the nucleic acids, and may comprise 1-8 M guanidine hydrochloride, 10-100 mM tris hydrochloride, 10-500 mM sodium chloride and 10-90% alcohol (isopropyl alcohol or ethyl alcohol). Accordingly, in the first removal step (S16), impurities other than the nucleic acids are removed from the magnetic particles.

Referring to FIG. 14, Embodiment 4 comprises a second discharge step (S17).

Referring to FIG. 5, in the second discharge step (S17), the mixture with the washing solution is sucked in the pipette 141, 142 and is located above the waste bottle 450. Subsequently, referring to FIG. 2, as the piston 120 moves downward, a second discharge pressure is applied to the pipette 141, 142 so that the mixture with the washing solution is discharged from the pipette 141, 142 to the waste bottle 450. At the same time, a magnetic field is applied to the pipette 141, 142 by the magnet mounting units 191, 192 so that the magnetic particles and the nucleic acids attached to the magnetic particles are not discharged from the pipette 141, 142 but remain in the pipette 141, 142. Accordingly, in the second discharge step (S17), the mixture with the washing solution excluding the magnetic particles and the nucleic acids attached thereto is discharged to the waste bottle 450.

Referring to FIG. 14, Embodiment 4 comprises a second removal step (S18). In the second removal step (S18), alcohol that may remain on the magnetic particles during the washing procedure is removed.

Referring to FIG. 5, in the second removal step (S18), the magnetic field is released and the magnetic particles and the nucleic acids attached thereto are injected to the high-temperature reaction tube 462 using the pipette 141, 142. As a result, the alcohol that remains on the magnetic particles is removed from the magnetic particles as it is heated and evaporated in the high-temperature reaction tube 462. The second removal step (S18) may comprise a step (S18-1) for injection into the pipette, a step (S18-2) for injection into the high-temperature reaction tube, and a step (S18-3) for air inflow/outflow.

Referring to FIG. 2, in the step (S18-1) for injection into the pipette, in the state where the magnetic particles are held in the pipette 141, 142, alcohol held in a unit well G (see FIG. 5) of the multi-well plate kit 420′ is injected into the pipette 141, 142 as the piston 121, 122 moves upward. The step (S18-1) for injection into the pipette is to mix the magnetic particles and the nucleic acids attached thereto with the alcohol held in the unit well G so that they may be easily injected to the high-temperature reaction tube 462.

Referring to FIG. 5, in the step (S18-2) for injection into the high-temperature reaction tube, the alcohol injected in the step (S18-1) for injection into the pipette is injected to the high-temperature reaction tube 462 along with the magnetic particles, the nucleic acids attached thereto and the alcohol from the washing solution remaining on the magnetic particles.

Referring to FIG. 2, in the step (S18-3) for air inflow/outflow, in the state where the magnetic particles, the nucleic acids attached thereto, the alcohol from the washing solution remaining on the magnetic particles, and the alcohol injected in the step (S18-1) for injection into the pipette are held in the high-temperature reaction tube 462, air is flown into and out of the high-temperature reaction tube 462 by the upward and downward movement of the piston 121, 122. By flowing air into and out of the high-temperature reaction tube 462 or heating the high-temperature reaction block or by performing both of them, the alcohol from the washing solution remaining on the magnetic particles and the alcohol injected in the step (S18-1) for injection into the pipette may be completely removed from the high-temperature reaction tube 462.

Referring to FIG. 14, Embodiment 4 comprises a nucleic acid separation step (S19).

Referring to FIG. 5, in the nucleic acid separation step (S19), a nucleic acid eluent held in a unit well F of the multi-well plate kit 420 is injected into the high-temperature reaction tubes 462 using the pipette 141, 142. As a result, the nucleic acids are separated from the magnetic particles in the high-temperature reaction tubes 462.

Referring to FIG. 14, Embodiment 4 comprises a nucleic acid collection step (S20).

Referring to FIG. 5, in the nucleic acid collection step (S20), the nucleic acid eluent containing the nucleic acids separated from the magnetic particles and the magnetic particles are sucked in the pipette 141, 142 and are located above the sample storage tube 442. Subsequently, referring to FIG. 2, as the piston 120 moves downward, a third discharge pressure is applied to the pipette 141, 142 so that the nucleic acid eluent containing the nucleic acids separated from the magnetic particles and the magnetic particles are discharged from the pipette 141, 142 to the sample storage tube 442. At the same time, a magnetic field is applied to the pipette 141, 142 by the magnet mounting units 191, 192 so that the magnetic particles are not discharged from the pipette 141, 142 but remain in the pipette 141, 142. Accordingly, in the nucleic acid collection step (S20), the nucleic acid eluent containing the nucleic acids excluding the magnetic particles is collected in the sample storage tube 442.

When the nucleic acids are separated from the biological samples and purified using the magnetic particles, the impurities other than the nucleic acids bound to the magnetic particles should be removed completely before the nucleic acid separation step (S19) using the nucleic acid eluent. For this purpose, in the first removal step (S16), the magnetic particles are washed using the washing solution comprising 10-90% alcohol.

However, if the alcohol remaining on the magnetic particles after the first removal step (S16) is eluted together with the nucleic acids during the elution using the nucleic acid eluent, direct or indirect reactions may occur with the enzymes used for polymerase chain reaction, real-time polymerase chain reaction, sequencing reaction, etc. This may result in decreased activity and sensitivity of the enzymes. Accordingly, the alcohol remaining on the magnetic particles should be completely removed before the elution of the nucleic acids using the nucleic acid eluent. Thus, Embodiment 4 comprises the second removal step (S18) in order to remove the alcohol remaining on the magnetic particles.

### Test Example 2

Chromosomal DNA was extracted from whole blood (200 μL) of a healthy person using Bioneer's Genomic DNA Extraction Kit (K-3032). The extraction was carried out according to the manufacturer's instructions. Final elution volume of the nucleic acid was 50 μL. Further, chromosomal DNA was extracted from the same volume of whole blood from the same person according to the procedure of Embodiment 4. After the nucleic acid extraction was completed, for thus obtained four samples, GAPDH gene application was carried out using primer and probe set designed to amplify and quantify human GAPDH gene and using Bioneer's real-time PCR kit (AccuPower Dualstar™ qPCR Premix, K-6100) and real-time quantitative PCR apparatus (Exicycler™ 96 Real-Time Quantitative Thermal Block, A-2060).

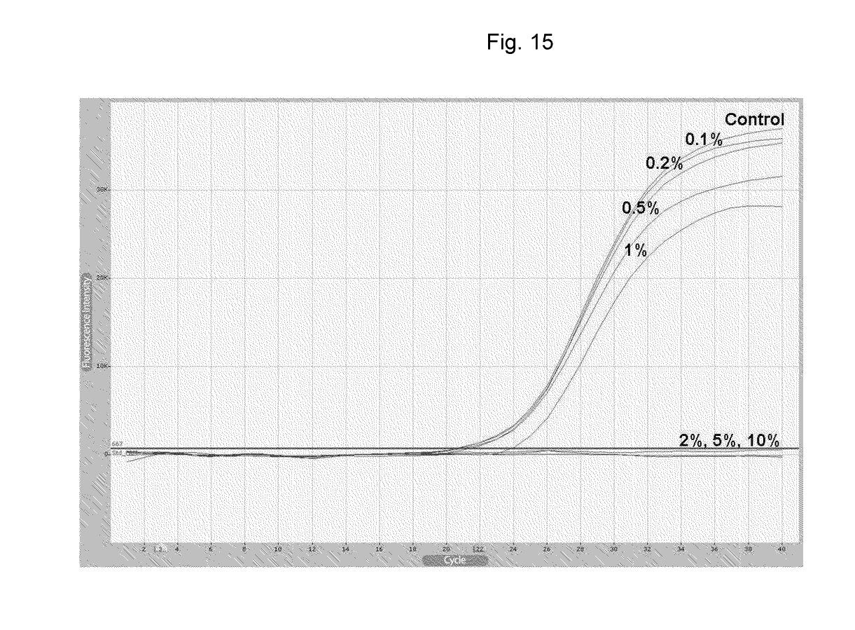

FIG. 15 is a graph showing a result of performing real-time PCR with different concentrations of ethyl alcohol.

Referring to FIG. 15, the fluorescence intensity started to decrease when alcohol concentration was 0.2% of the total volume. When the concentration exceeded 2%, the experiment was meaningless.

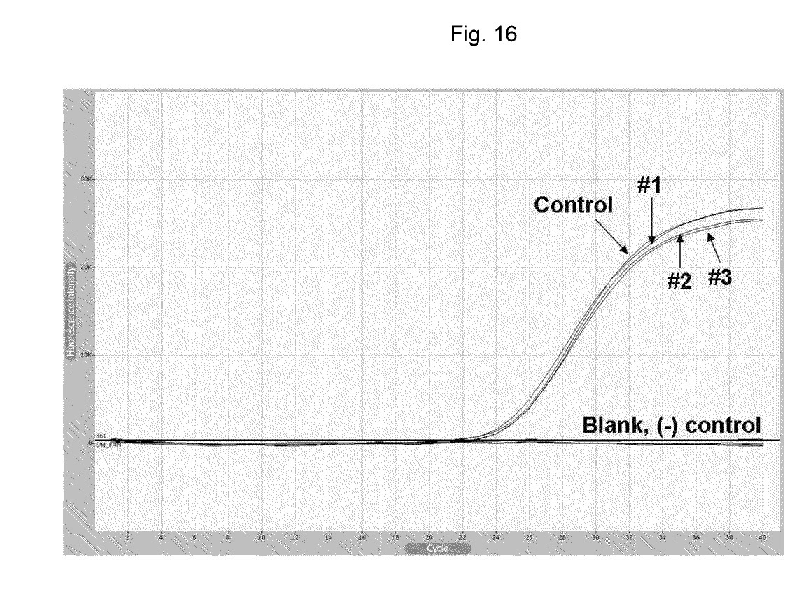

FIG. 16 is a graph showing a result of performing real-time polymerase chain reaction using DNAs extracted according to Embodiment 4 (#1, #2, #3). Control is the result of carrying out real-time PCR for the DNA extracted using a commercially available kit for chromosomal DNA extraction (Bioneer, K-3032). (−) control is the result when sterilized triple distilled water was added instead of template DNA in Control experiment. Blank is the result when only sterilized distilled water was added. It can be seen from FIG. 16 that DNA was separated purely according to Embodiment 4.

### INDUSTRIAL APPLICABILITY

The present invention is widely applicable in genetic engineering, medical industry and other fields since it allows automatic separation of nucleic acids, proteins, or the like from biological samples using magnetic particles.

1. An automatic refining apparatus for separating target materials from a plurality of biological samples by using magnetic particles to which the magnetic particles are to be reversibly coupled, comprising:
a pipette block having a plurality of pipettes mounted in at least two rows for sucking and discharging biological samples including target materials into and out of the plurality of pipettes;
a fixing body supporting the pipette block;
a magnetic field application unit for applying and releasing a magnetic field to the pipettes of each row mounted on the pipette block;
a pipette block upward/downward moving means moving the pipette block upward and downward; and
a pipette block forward/backward moving means moving the pipette block forward and backward.

2. The automatic refining apparatus according to claim 1, wherein the pipette block comprises:
a piston fixing plate wherein a plurality of pistons are provided in two rows;
a piston moving means moving the piston fixing plate upward and downward;
a piston guiding unit having piston guide holes guiding the upward and downward movement of the a plurality of pistons; and
pipette mounting units extending below the piston guiding unit in two rows so as to be engaged with the inner periphery of the plurality of pipettes arranged in two rows and having a plurality of connecting holes respectively communicating with the piston guide holes.

3. The automatic refining apparatus according to claim 2, wherein engagement rings are provided at the outer periphery of the pipette mounting units so that the pipette mounting units may be engaged with the inner periphery of the pipettes.

4. The automatic refining apparatus according to claim 2, wherein the pipette block comprises:
a piston guiding unit supporting plate supporting the piston guiding unit;
a guide rod protruding from an upper surface of the piston guiding unit supporting plate and guiding the upward and downward movement of the piston fixing plate; and
a pipette separating unit contacting a lower surface of the piston fixing plate and separating the plurality of pipettes mounted on the pipette mounting units.

5. The automatic refining apparatus according to claim 4, wherein the pipette separating unit comprises:
a detachable upper plate provided above the piston guiding unit through which the plurality of pistons penetrate;
a detachable lower plate provided below the piston guiding unit supporting plate, allowing the pipette mounting units to penetrate through, and separating the plurality of pipettes mounted on the pipette mounting units by pressing the upper end thereof downward;
an up/down connecting rod connecting the detachable upper plate and the detachable lower plate with a gap;
a protruding rod provided on an upper surface of the detachable lower plate and protruding above the piston guiding unit supporting plate via through-holes formed on the piston guiding unit supporting plate; and
a spring the lower end of which being supported by the upper surface of the piston guiding unit supporting plate and the upper end of which being supported by an upper end of the protruding rod so as to exert an elastic force so that the detachable lower plate is fastened to the piston guiding unit supporting plate.

6. The automatic refining apparatus according to claim 2, wherein the piston moving means comprises:
a piston driving motor supporting plate supported by the guide rod and having a piston driving motor mounted thereon; and
a piston control screw moving upward and downward by the piston driving motor and the lower end of which being connected to the piston fixing plate.

7. The automatic refining apparatus according to claim 1, wherein the magnetic field application unit comprises:
a first row magnet mounting unit having a magnet for applying a magnetic field to the pipettes mounted on the first row of the pipette block;
a second row magnet mounting unit having a magnet for applying a magnetic field to the pipettes mounted on the second row of the pipette block;
a first row magnet mounting unit moving means for controlling a distance between the magnet of the first row magnet mounting unit and the pipettes mounted on the first row of the pipette block; and
a second row magnet mounting unit moving means for controlling a distance between the magnet of the second row magnet mounting unit and the pipettes mounted on the second row of the pipette block,
wherein
the strength and duration of the magnetic field applied to the pipettes of the first row by the first row magnet mounting unit and the first row magnet mounting unit moving means are the same as the strength and duration of the magnetic field applied to the pipettes of the second row by the second row magnet mounting unit and the second row magnet mounting unit moving means.

8. The automatic refining apparatus according to claim 7, wherein the first row magnet mounting unit comprises a first row middle plate located by the first row magnet mounting unit moving means between neighboring pipettes among the pipettes of the first row and having a magnet mounted thereon and a first row end plate located by the first row magnet mounting unit moving means outside of a pipette located at the side end among the pipettes of the first row and having a magnet mounted thereon, and the second row magnet mounting unit comprises a second row middle plate located by the second row magnet mounting unit moving means between neighboring pipettes among the pipettes of the second row and having a magnet mounted thereon and a second row end plate located by the second row magnet mounting unit moving means outside of a pipette located at the side end among the pipettes of the second row and having a magnet mounted thereon.

9. The automatic refining apparatus according to claim 8, wherein the first row middle plate and the first row end plate have through-holes provided in a direction parallel to the row direction of the pipettes of the first row to allow the mounting of the magnets, and the second row middle plate and the second row end plate have through-holes provided in a direction parallel to the row direction of the pipettes of the second row to allow the mounting of the magnets.

10. The automatic refining apparatus according to claim 7, wherein the first row magnet mounting unit moving means comprises a first row gear connected to the pipette block and rotated by a magnet mounting unit motor and a first row rotation shaft rotated by the rotation of the first row gear, the second row magnet mounting unit moving means comprises a second row gear connected to the pipette block and rotated in the opposite direction of the first row gear as being engaged with the first row gear and a second row rotation shaft rotated by the rotation of the second row gear, and the first row magnet mounting unit is radially connected to the first row rotation shaft so as to rotate and the second row magnet mounting unit is radially connected to the second row rotation shaft so as to rotate.

11. The automatic refining apparatus according to claim 1, wherein the pipette block is mounted on the fixing body so as to be movable upward and downward, the pipette block upward/downward moving means comprises an up/down movement motor provided at the fixing body and an up/down movement screw rotated by the up/down movement motor so as to move a fixing nut fixed to the pipette block upward and downward, and the pipette block forward/backward moving means comprises a forward/backward movement supporting rod supporting the fixing body so as to be movable forward and backward and a forward/backward moving belt attached to the fixing body so as to move the fixing body forward and backward.

12. The automatic refining apparatus according to claim 1, which comprises a base plate below the fixing body, the base plate having a multi-well plate kit, a pipette rack insertably holding the plurality of pipettes mounted on the pipette block in two rows, a sample storage tube rack insertably holding a plurality of sample storage tubes for storing the purified sample in two rows, and a waste bottle for holding waste solution discarded from the plurality of pipettes mounted on the pipette block mounted thereon.

13. The automatic refining apparatus according to claim 12, wherein the base plate has a high-temperature reaction block for heating a plurality of high-temperature reaction tubes insertably held in two rows mounted thereon.

14. The automatic refining apparatus according to claim 12, which comprises a casing accommodating the pipette block, the fixing body, the pipette block upward/downward moving means, the pipette block forward/backward moving means and the base plate, wherein a UV lamp or an ozone generator for sterilization is provided in the casing.

15. A multi-well plate kit used in the automatic refining apparatus according to one of claim 1, comprising a plurality of unit wells arranged in two neighboring rows and a film sealing an upper end of the plurality of unit wells, wherein solutions for separation of target materials are contained in the unit wells excluding at least one unit well(s) such that the same solution is contained in the same unit well.

16. The multi-well plate kit according to claim 15, wherein the solution contained in one of the sealed unit wells is an aqueous suspension wherein magnetic particles are suspended and the magnetic particles suspended in the aqueous suspension are spherical magnetic particles coated with silica.

17. A method for extracting nucleic acids from biological samples using the automatic refining apparatus according to claim 1, comprising:
mixing a biological sample with a cell lysis solution contained in a well of the multi-well plate kit using the pipette;
mixing the sample mixed with the cell lysis solution with a coupling solution contained in a well of the multi-well plate kit using the pipette;
mixing the mixture with the coupling solution with an aqueous suspension of magnetic particles contained in a well of the multi-well plate kit using the pipette;
in the state where the mixture with the coupling solution is held in the pipette, applying a discharge pressure to the pipette such that the mixture is discharged from the pipette while applying a magnetic field to the pipette at the same time such that the magnetic particles and materials attached to the magnetic particles are not discharged by the discharge pressure but remain in the pipette;
releasing the magnetic field and mixing the magnetic particles and the materials attached to the magnetic particles with a washing solution containing alcohol contained in a well of the multi-well plate kit so as to remove impurities other than nucleic acids from the magnetic particles;
in the state where the mixture with the washing solution is held in the pipette, applying a discharge pressure to the pipette such that the mixture is discharged from the pipette while applying a magnetic field to the pipette at the same time such that the magnetic particles with nucleic acids attached thereto are not discharged by the discharge pressure but remain in the pipette;
releasing the magnetic field and injecting the magnetic particles with the nucleic acids attached thereto into a high-temperature reaction tube on a high-temperature reaction block so as to remove the alcohol from the washing solution remaining on the magnetic particles;
mixing a nucleic acid eluent contained in a well of the multi-well plate kit with the magnetic particles held in the high-temperature reaction tube using the pipette so as to separate the nucleic acids; and
in the state where the nucleic acid eluent including the nucleic acids separated from the magnetic particles and the magnetic particles are held in the pipette, applying a discharge pressure to the pipette such that the nucleic acid eluent including the nucleic acids is discharged from the pipette while applying a magnetic field to the pipette at the same time such that the magnetic particles are not discharged by the discharge pressure but remain in the pipette.

18. A method for extracting nucleic acids from biological samples using the automatic refining apparatus according to claim 13, comprising:
mixing a biological sample contained in a well of the multi-well plate kit with a cell lysis solution contained in a well of the multi-well plate kit using the pipette;
mixing the cell lysis solution and the biological sample with lysed cells with a coupling solution contained in a well of the multi-well plate kit using the pipette;
mixing the mixture with the coupling solution with an aqueous suspension of magnetic particles contained in a well of the multi-well plate kit using the pipette;
in the state where the mixture with the coupling solution is held in the pipette and located above the waste bottle, applying a discharge pressure to the pipette by a downward movement of a piston such that the mixture with the coupling solution is discharged from the pipette while applying a magnetic field to the pipette at the same time using a magnet mounting unit such that the magnetic particles and materials attached to the magnetic particles are not discharged by the discharge pressure but remain in the pipette;
releasing the magnetic field and mixing the magnetic particles and the materials attached to the magnetic particles with a washing solution containing alcohol contained in a well of the multi-well plate kit so as to remove impurities other than nucleic acids from the magnetic particles;
in the state where the mixture with the washing solution is held in the pipette and located above the waste bottle, applying a discharge pressure to the pipette by a downward movement of the piston such that the mixture with the washing solution is discharged from the pipette while applying a magnetic field to the pipette at the same time using the magnet mounting unit such that the magnetic particles with nucleic acids attached thereto are not discharged by the discharge pressure but remain in the pipette;
releasing the magnetic field and injecting the magnetic particles with the nucleic acids attached thereto into a high-temperature reaction tube so as to remove the alcohol from the washing solution remaining on the magnetic particles;
mixing a nucleic acid eluent contained in a well of the multi-well plate kit with the magnetic particles held in the high-temperature reaction tube using the pipette so as to separate the nucleic acids; and
in the state where the nucleic acid eluent including the nucleic acids separated from the magnetic particles and the magnetic particles are held in the pipette and located above the sample storage tube, applying a discharge pressure to the pipette by a downward movement of the piston such that the nucleic acid eluent including the nucleic acids is discharged from the pipette while applying a magnetic field to the pipette at the same time using the magnet mounting unit such that the magnetic particles are not discharged by the discharge pressure but remain in the pipette.

19. The method for extracting nucleic acids from biological samples according to claim 18, wherein said removing the alcohol from the washing solution remaining on the magnetic particles comprises:
in the where the magnetic particles are held in the pipette, injecting alcohol contained in a well of the multi-well plate kit to the pipette by an upward movement of the piston so as to allow easy injection of the magnetic particles into the high-temperature reaction tube; and
injecting the alcohol injected from the well of the multi-well plate kit to the pipette to the high-temperature reaction tube along with the magnetic particles with the nucleic acids thereto.

20. The method for extracting nucleic acids from biological samples according to claim 19, wherein said removing the alcohol from the washing solution remaining on the magnetic particles comprises, in the where the magnetic particles with the nucleic acids thereto and the alcohol injected from the well of the multi-well plate kit to the pipette are held in the high-temperature reaction tube, flowing in or out air by heating the high-temperature reaction block or by an upward or downward movement of the piston or both.

21. The method for extracting nucleic acids from biological samples according to claim 17, which comprises, before mixing the biological sample with the coupling solution contained in a unit well of the multi-well plate kit, injecting the biological sample mixed with the cell lysis solution to the high-temperature reaction tube using the pipette so as to allow easy cell lysis of the biological sample.
